(19) United States
(12) Patent Application Publication
(10) Pub. No.: US 2024/0117019 A1
(43) Pub. Date: Apr. 11, 2024

(54) HUMAN MONOCLONAL ANTIBODIES AGAINST PNEUMOCOCCAL ANTIGENS

(71) Applicant: University of Georgia Research Foundation, Inc., Athens, GA (US)

(72) Inventors: Jarrod Mousa, Athens, GA (US); Aaron Gingerich, Athens, GA (US); Jiachen Huang, Athens, GA (US); Fredejah Royer, Athens, GA (US); Fikri Y. Avci, Watkinsville, GA (US)

(73) Assignee: University of Georgia Research Foundation, Inc., Athens, GA (US)

(21) Appl. No.: 18/264,733

(22) PCT Filed: Feb. 7, 2022
(86) PCT No.: PCT/US2022/015459

§ 371 Date: Aug. 8, 2023

Related U.S. Application Data
(60) Provisional application No. 63/147,393, filed on Feb. 9, 2021.

(51) Int. Cl. C07K 16/12; A61P 31/04; G01N 33/569

(52) U.S. Cl. CPC C07K 16/1275; A61P 31/04; G01N 33/56944; C07K 2317/14; C07K 2317/31; C07K 2317/565; C07K 2317/567; C07K 2317/94

(57) ABSTRACT

Monoclonal antibodies and antigen binding fragments that specifically bind pneumococcal proteins, specifically pneumococcal histidine triad protein (PhtD) and pneumococcal surface protein A (PspA), are provided. Also disclosed are nucleic acid molecules encoding the antibody, antigen binding fragment, a V_{H} or V_{L} of the antibody, or a multipiece antibody including the V_{H} and/or V_{L}, vectors including these nucleic acid molecules, and host cells transfected with these vectors. Methods of inhibiting, treating, and detecting a Streptococcus pneumoniae infection are also provided.

### CROSS-REFERENCE TO RELATED APPLICATION

This claims the benefit of U.S. Provisional Application No. 63/147,393, filed Feb. 9, 2021, herein incorporated by reference in its entirety.

### ACKNOWLEDGMENT OF GOVERNMENT SUPPORT

This invention was made with government support under grant 1K01OD026569, R01AI123383, and GM109435 awarded by the National Institutes of Health and grant UL1TR002378 from the National Center for Advancing Translation Sciences (NCATS); the government has certain rights in the invention.

### FIELD OF THE DISCLOSURE

This relates to monoclonal antibodies and antigen binding fragments that specifically bind Streptococcus pneumoniae, for example that specifically bind to pneumococcal histidine triad protein (PhtD) or pneumococcal surface protein A (PspA), and their use for inhibiting and detecting a Streptococcus pneumoniae infection.

### BACKGROUND

Streptococcus pneumoniae is a leading cause of infectious morbidity and mortality despite the widespread use of two vaccines for disease prevention (Levine et al. (2006) Lancet 367:1880-1882). The World Health Organization estimates over 1 million deaths occur worldwide each year due to pneumococcal infection (World Health Organization (2003) Wkly Epidemiol Rec 78:110-119). Similar to other respiratory pathogens, individuals below the age of 2 and above 65 years of age are more susceptible to invasive pneumococcal disease (Practices AC on I. (2000) MMWR Recomm Rep 49:1-35). In addition, there is also an increased frequency and risk of severe infection in individuals with preexisting conditions, such as those with diabetes, chronic obstructive pulmonary disease, cardiovascular diseases, and human immunodeficiency virus (van der Poll and Opal (2009) Lancet 374:1543-1556). Although vaccination is widespread in the developed world, pneumococcal infection is responsible for 30% of adult pneumonia and has a mortality rate of 11-40% (Bridy-Pappas et al. (2005) Pharmacotherapy 25:1193-212). Furthermore, in regions of the world with high childhood mortality rates, pneumococcal pneumonia is the cause of death for 20-50% of children (Williams et al. (2002) Lancet Infect Dis 2:25-32).

Streptococcus pneumoniae is a common resident of the upper respiratory tract, and pneumococcal carriage precedes active infection (Sulikowska et al. (2004) J Clin Microbiol 42:3942-3949; Simell et al. (2012) Expert Rev Vaccines 11:841-855). In young children, carriage rates of Streptococcus pneumoniae can be as high as 40-60% (Cardozo et al. (2008) J Med Microbiol 57:185-189). Colonization is typically asymptomatic; however, Streptococcus pneumoniae can rapidly disseminate, often following a primary infection such as influenza, to cause pneumonia and invasive disease. Repeated colonization with Streptococcus pneumoniae typically results in immunization, and several studies have determined that colonization induces serum antibody responses to the capsular polysaccharide, and both serum antibody and cellular immune responses to protein antigens (Weinberger et al. (2008) J Infect Dis 197:1511-1518; Goldblatt et al. (2005) J Infect Dis 192:387-93; McCool et al. (2002) J Exp Med 195:359-365; Prevaes et al. (2012) Infect Immun 80:2221-2186-2193; Turner et al. (2013) Clin Microbiol Infect 19:E551-E558; Zhang et al. (2006) Eur J Immunol 36:46-57; Mureithi et al. (2009) J Infect Dis 200:783-793; and Wright et al. (2013) PLoS Path e1003274). These antibody levels in serum increase during the first few years of life, but tend to decrease in the elderly, which may contribute to the higher risk of disease observed in children and the elderly (Simell et al. (2008) Clin Vacc Immunol 15:1391-1397).

Although vaccines have been effective at reducing the incidence of pneumococcal disease, a rise in the incidence of non-vaccine serotypes has occurred, termed serotype replacement (Wantuch et al. (2018) Hum Vaccines Immunother 14:2303-2309). In addition, the incidence of invasive disease due to serotypes 3 and 19A have persisted despite widespread vaccination (Linley et al. (2019) Vaccines 7: doi: 10.3390/vaccines7010004). In terms of treatment, antibiotic resistance among non-vaccine serotypes has risen, and presents challenges in treating pneumococcal infection (Lo et al. (2019) Lancet Infect Dis 19:759-769). Based on the limitations of current vaccines and treatments, additional treatment options are greatly needed.

### SUMMARY OF THE DISCLOSURE

Isolated monoclonal antibodies and antigen binding fragments that specifically bind to Streptococcus pneumoniae are provided. In some embodiments, the antibody or antigen binding fragment includes one of:
a) a heavy chain variable region (V_{H}) and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as SEQ ID NOs: 1 and 5, respectively;
b) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 9 and 13, respectively;
c) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 17 and 21, respectively;
d) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 25 and 29, respectively;
e) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 33 and 37, respectively.

In further embodiments, disclosed are multi-specific antibodies that include combinations of 2 or more these antibodies and/or antigen binding fragments.

Also disclosed are nucleic acid molecules encoding the antibody, antigen binding fragment, a V_{H} or V_{L} of the antibody, or a multipiece antibody including the V_{H} and/or V_{L}, vectors including these nucleic acid molecules, and host cells transfected with these vectors.

In more embodiments, methods are disclosed for inhibiting a Streptococcus pneumoniae infection in a subject.

In further embodiments, methods are disclosed for detecting Streptococcus pneumoniae in a biological sample.

The foregoing and other features and advantages of the invention will become more apparent from the following detailed description of several embodiments which proceeds with reference to the accompanying figures.

### BRIEF DESCRIPTION OF THE FIGURES

FIG. 1. Purification of recombinant PhtD and PspA proteins. SDS-PAGE (left) and western blot (right) of purified recombinantly expressed PspA and PhtD. Both proteins were pure with the appearance of degradation products.

FIG. 2. ELISA binding responses from the supernatant of stimulated B cells to recombinant PhtD and PspA proteins.

FIGS. 3A-3B. Monoclonal antibody (mAb) binding to PhtD and PspA. FIG. 3A shows ELISA binding curves of anti-PhtD mAbs against recombinant PhtD protein. PspA16 was utilized as a negative control. > indicates no binding was observed at an OD₄₀₅ over 1 Abs at the highest concentration. FIG. 3B shows binding of PspA16 to recombinant PspA. For FIGS. 3A and 3B, computed EC₅₀ values in ng/mL are reported from a non-linear regression curve fit (agonist). Data points indicate the average of four replicates from one of at least two independent experiments. Error bars indicate 95% confidence intervals.

FIG. 4. SDS-PAGE of the purified maltose binding protein (MBP) PhtD fragment fusion proteins. Each fusion protein was pure after purification, with the exception of free MBP.

FIG. 5. ELISA binding curves of the PhtD mAbs to each MBP-PhtD fragment. A summary of the binding curves is displayed below the binding curves. Data points indicate the average of four replicates from one of at least two independent experiments. Error bars indicate 95% confidence intervals.

FIG. 6. Epitope mapping of the PhtD-specific mAbs. Data indicate the percent binding of the competing antibody in the presence of the primary antibody, compared with the competing antibody alone. Cells filled in black indicate full competition, in which <33% of the uncompeted signal was observed; cells in gray indicate intermediate competition, in which the signal was between 33% and 66%; and cells in white indicate noncompetition, where the signal was >66%.

FIG. 7. SDS-PAGE of recombinant MBP PspA fragment fusion proteins. Fragments 1-2 and 4-5 purified well, with only visible MBP protein as a contaminant. PspA fragment 3 has multiple co-purified bands and/or degradation products.

FIG. 8. ELISA binding curves for PspA16 to each fragment. PspA16 bound to fragment 1 and fragment 4, but not others, suggesting the epitope lies within amino acids 1-247. Data points indicate the average of four replicates from one of at least two independent experiments. Error bars indicate 95% confidence intervals.

FIG. 9. Western blot of TIGR4 and TCH8431 strains with PspA16 and PhtD3 as the primary antibodies. In the western blot for PspA16, PspA fragment 1-438 fused to the MBP was used as the positive control, and MBP was used as the negative control. In the PhtD3 western blot, recombinant PhtD was used as the positive control, and E. coli lysates were used as the negative control.

FIG. 10. ELISA binding curves for the isolated mAbs against plates coated with fixed bacteria. Data represent the average of four data points from one of at least two independent experiments, and error bars are the standard deviation.

FIG. 11. Heat map and percentages for antibody binding to each pneumococcal serotype. Data are averages from 3-4 experiments, and are the percent of bacteria that are APC-positive. MPV314 and MPV414 are human antibodies specific to the human metapneumovirus fusion protein, and these were used as negative controls.

FIGS. 12A-12F. Protective efficacy of anti-PhtD mAbs. FIG. 12A: ELISA binding curve of mAbs PhtD3 and PhtD8, the isotype-switched mAbs (PhtD3-IgG₂ₐ and PhtD8-IgG₂ₐ), and an IgG₂ₐ isotype control. FIG. 12B: Prophylactic efficacy of mAb PhtD3-IgG₂ₐ in an intranasal infection model of pneumococcal serotype 3 (strain WU2) in C57BL/6 mice. **, P=0.0012, ns=not significant via log-rank (Mantel-Cox) test. n=10 mice/group. FIG. 12C: Prophylactic efficacy of mAb PhtD8-IgG₂ₐ in an intranasal infection model of pneumococcal serotype 3 (strain WU2) in C57BL/6 mice. ***, P=0.0009, ns=not significant via log-rank (Mantel-Cox) test. n=10 mice/group. FIG. 12D: Prophylactic efficacy of mAb PhtD3-IgG₂ₐ in an intranasal infection model of pneumococcal serotype 4 (strain TIGR4) in CBA/N mice. **, P=0.0045 via log-rank (Mantel-Cox) test. n=15 mice/group. FIG. 12E: Prophylactic efficacy of mAb PhtD3-IgG₂ₐ in an intravenous infection model of pneumococcal serotype 4 (strain TIGR4) in C57BL/6 mice. **P=0.0101 via log-rank (Mantel-Cox) test. n=13-15 mice/group. FIG. 12F: Treatment efficacy of mAb PhtD3-IgG2a in an intranasal infection model of pneumococcal serotype 3 (strain WU2) in C57BL/6 mice. ***P=0.0002, ns=not significant via log-rank (Mantel-Cox) test. n=20 mice/group.

FIGS. 13A-13C. Protective efficacy of anti-PhtD and anti-PspA mAbs. FIG. 13A: Survival curve of mice that were administered PhtD7 (15 mg/kg) or isotype control IgG2a (15 mg/kg) via intraperitoneal injection two hours prior to intranasal infection with Streptococcus pneumoniae (5×10⁶ CFU of serotype 3 (WU2)). Survival of mice was monitored over time and expressed as a percent. Statistical comparison of survival curves using log-ranked (Mantel-Cox) test showed a ***p-value of 0.006. FIG. 13B: Survival curve of mice that were administered PspA16 (15 mg/kg), isotype control IgG₂ₐ (15 mg/kg), or PBS via intraperitoneal injection two hours prior to intranasal infection with Streptococcus pneumoniae (5×10⁶ CFU of serotype 3 (WU2)). Survival of mice was monitored over time and expressed as a percent. Statistical comparison of survival curves using log-ranked (Mantel-Cox) test showed a **p-value of 0.0071.

FIG. 13C: Survival curve of mice that were administered a combination of PhtD3-IgG₂ₐ and PhtD7 (7.5 mg/kg each) or isotype control (15 mg/kg) via intraperitoneal injection 24 hours post intranasal infection with Streptococcus pneumoniae (5×10⁶ CFU of serotype 3 (WU2)). Survival of mice was monitored over time and expressed as a percent. Statistical comparison of survival curves using log-ranked (Mantel-Cox) test showed a ****p-value of <0.0001.

FIGS. 14A-14B. Opsonophagocytic activity of PhtD-specific human mAbs. FIG. 14A: mAbs and serum were tested in a standard opsonophagocytic killing assay (OPKA) using differentiated HL-60 cells. Bacteria of the indicated serotype (serotype 4, serotype 3, or serotype 19A) were opsonized with antibodies, and subsequently HL-60 cells were added before plating onto blood agar plates. Plates were incubated overnight and CFUs counted. Data are averages of three replicates from one experiment. Error bars represent the range. % Bacterial Killing was calculated as the counted CFU value of each triplicate normalized against the average of the No Ab control. One-way ANOVA analysis with Dunnett's multiple comparisons test was used to determine significance. ns=not significant, ****P<0.0001. FIG. 14B: mAbs and serum were tested in a flow-based opsonophagocytosis assay. pHRodo™-labeled bacteria of the indicated serotype (serotype 4, serotype 3, or serotype 19A) were opsonized with antibodies, and incubated with HL-60 cells before being subjected to analysis by flow cytometry. Data indicate the percent of CD38+CD11b+HL-60 cells that are pHRodo™+. Each bar graph is the average of three experimental replicates and error bars are the standard deviation. ns=not significant, ***P=0.0001-0.0006, ****P<0.0001 via one-way ANOVA analysis with Dunnett's multiple comparisons test. MPV414 is a human mAb specific to the human metapneumovirus fusion protein.

FIG. 15. Lung and blood bacterial titers three days post infection of mice treated with PhtD3-IgG2a (labeled as PhtD3) or isotype. The data is shown as log₁₀ of the CFU/mL of lung homogenates or blood. A statistical comparison for lung and blood titers using an unpaired t-test showed a p-value of 0.0002 and 0.009, respectively.

FIG. 16. Survival curve of PhtD3-IgG2a (labeled as PhtD3) treated mice in a co-infection model with influenza and Streptococcus pneumoniae. The data shows survival following primary infection with influenza (or S. pneumoniae for the “Spn only” treatment). The dotted line at day 7 (labeled “Spn”) marks time of intranasal co-infection with S. pneumoniae for PhtD3, isotype, and PBS treatments. Statistical comparison of survival curves of PhtD3 vs isotype control groups using log-ranked (Mantel-Cox) test showed a p-value of <0.0001.

### SEQUENCES

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named “Sequence Listing.txt,” created on Jan. 13, 2022, 28, 672 bytes, which is incorporated by reference herein. In the accompanying sequence listing:

SEQ ID NO: 1 is the amino acid sequence of the PhtD3 V_{H}.

SEQ ID NOs: 2, 3, and 4 are the amino acid sequences of the HCDR1, HCDR2, and HCDR3, respectively, of the PhtD3 V_{H}.

SEQ ID NO: 5 is the amino acid sequence of the PhtD3 V_{L}.

SEQ ID NOs: 6, 7, and 8 are the amino acid sequences of the LCDR1, LCDR2, and LCDR3, respectively, of the PhtD3 V_{L}.

SEQ ID NO: 9 is the amino acid sequence of the PhtD8 V_{H}.

SEQ ID NOs: 10, 11 and 12 are the amino acid sequences of the HCDR1, HCDR2, and HCDR3, respectively, of the PhtD8 V_{H}.

SEQ ID NO: 13 is the amino acid sequence of the PhtD8 V_{L}.

SEQ ID NOs: 14, 15, and 16 are the amino acid sequences of the LCDR1, LCDR2, and LCDR3, respectively, of the PhtD8 V_{L}.

SEQ ID NO: 17 is the amino acid sequence of the PhtD6 V_{H}.

SEQ ID NOs: 18, 19, and 20 are the amino acid sequences of the HCDR1, HCDR2, and HCDR3, respectively, of the PhtD6 V_{H}.

SEQ ID NO: 21 is the amino acid sequence of the PhtD6 V_{L}.

SEQ ID NOs: 22, 23, and 24 are the amino acid sequences of the LCDR1, LCDR2, and LCDR3, respectively, of the PhtD6 V_{L}.

SEQ ID NO: 25 is the amino acid sequence of the PhtD7 V_{H}.

SEQ ID NOs: 26, 27, and 28 are the amino acid sequences of the HCDR1, HCDR2, and HCDR3, respectively, of the PhtD7 V_{H}.

SEQ ID NO: 29 is the amino acid sequence of the PhtD7 V_{L}.

SEQ ID NOs: 30, 31, and 32 are the amino acid sequences of the LCDR1, LCDR2, and LCDR3, respectively, of the PhtD7 V_{L}.

SEQ ID NO: 33 is the amino acid sequence of the PspA16 V_{H}.

SEQ ID NOs: 34, 35, and 36 are the amino acid sequences of the HCDR1, HCDR2, and HCDR3, respectively, of the PspA16 V_{H}.

SEQ ID NO: 37 is the amino acid sequence of the PspA16 V_{L}.

SEQ ID NOs: 38, 39, and 40 are the amino acid sequences of the LCDR1, LCDR2, and LCDR3, respectively, of the PspA16 V_{L}.

SEQ ID NO: 41 is a nucleic acid sequence encoding PhtD3 V_{H}.

SEQ ID NO: 42 is a nucleic acid sequence encoding PhtD3 V_{L}.

SEQ ID NO: 43 is a nucleic acid sequence encoding PhtD8 V_{H}.

SEQ ID NO: 44 is a nucleic acid sequence encoding PhtD8 V_{L}.

SEQ ID NO: 45 is a nucleic acid sequence encoding PhtD6 V_{H}.

SEQ ID NO: 46 is a nucleic acid sequence encoding PhtD6 V_{L}.

SEQ ID NO: 47 is a nucleic acid sequence encoding PhtD7 V_{H}.

SEQ ID NO: 48 is a nucleic acid sequence encoding PhtD7 V_{L}.

SEQ ID NO: 49 is a nucleic acid sequence encoding PspA16 V_{H}.

SEQ ID NO: 50 is a nucleic acid sequence encoding PspA16 V_{L}.

SEQ ID NOs: 51-64 are nucleic acid sequences of primers.

### DETAILED DESCRIPTION OF SEVERAL EMBODIMENTS

Monoclonal antibodies that specifically bind Streptococcus pneumoniae, such as histidine triad protein (PhtD) and pneumococcal surface protein A (PspA), are disclosed herein. PhtD is a highly conserved surface protein on S. pneumoniae, varying 91-98% among strains isolated from invasive disease cases in children (Yun et al. (2015) PLoS One 10:e0134055). One study of 107 pneumococcal strains showed PhtD was expressed in 100% of tested serotypes, while other studies have found that PhtD is widely prevalent, but absent in a subset of isolated strains (Rioux et al. (2011) Microbiology 157:335-348; Kawaguchiya et al. (2019) Pathog 8:162; Blumental et al. (2015) PLoS One 10:e0133885; and Rioux et al. (2011) Microbiology 157:336-348). The function of the Pht family of proteins has not been fully elucidated, although data has implicated the proteins are involved in attachment of S. pneumoniae to respiratory epithelial cells (Plumptre et al. (2013) Infect Immun 2013/07/22. 81:3644-3651; and Kallio et al. (2014) Infect Immun 2014/02/03. 82:1683-1691).

PhtD is immunogenic and induces protective humoral immunity, and vaccination with PhtD protein has been shown to reduce colonization, sepsis, and pneumonia (Adamou et al. (2001) Infect Immun 69:949-958; Godfroid et al. (2011) Infect Immun 79:238 LP-245; and Wizemann et al. (2001) Infect Immun 69:1593-1598). PhtD has also been shown to protect against systemic pneumococcal disease in a mouse model, and immunization of rhesus macaques with PhtD along with detoxified pneumolysin protected the animals against pneumococcal infection (Adamou et al. (2001) Infect Immun 69:949-958; and Denoël et al. (2011) Vaccine 29:5495-5501).

All Pht proteins are immunogenic and induce protective humoral immunity, and vaccination with these proteins was shown to reduce colonization, sepsis, and pneumonia (Adamou et al. (2001) Infect Immun 69:949-958; Godfroid et al. (2011) Infect Immun 79:238 LP-245; and Wizemann et al. (2001) Infect Immun 69:1593-1598). PhtD has been shown to protect against systemic pneumococcal disease in a mouse model, and immunization of rhesus macaques with PhtD along with detoxified pneumolysin protected the animals against pneumococcal infection (Adamou et al. (2001) Infect Immun 69:949-958; and Denoël et al. (2011) Vaccine 29:5495-5501). Fragments of PhtD have also been assessed for protective efficacy, and somewhat conflicting reports have demonstrated that both the N and C terminus are immunogenic and protective (Plumptre et al. (2013) PLoS One 8:e78916; André et al. (2020) Vaccine 38:4146-4153). PhtD was recently used as an antigen in a phase IIb clinical trial, demonstrating that PhtD remains an antigen of interest in pneumococcal vaccinology, although PhtD was administered along with PCV13, so a direct comparison of PhtD vs PCV13 was not accomplished. Mouse monoclonal antibodies to PhtD were shown to protect mice using a macrophage and complement dependent mechanism, and human polyclonal antibodies to PhtD were shown to reduce adherence of the pneumococcus to lung epithelial cells and reduce murine nasopharyngeal colonization (Visan et al. (2018) Hum Vaccin Immunother 14:489-494). Human polyclonal antibodies generated in response to alum adjuvanted PhtD vaccination were also shown to protect mice from disease (Brookes et al. (2015) Hum Vaccin Immunother 11:1836-1839).

PspA, is another important virulence factor of S. pneumoniae and one of the most abundant surface proteins (Rosenow et al. (1997) Mol Microbiol 25:819-829). As with PhtD, PspA is found in the majority of examined clinical isolates (Hollingshead et al. (2006) J Med Microbiol 55:215-221). PspA mutant strains are cleared faster from the blood of mice compared to intact strains, and vaccination with PspA protein protects mice from pneumococcal infection (Briles et al. 1988. Rev Infect Dis 10 Suppl. 2:S372-4; Bosarge et al. (2001) Infect Immun 69:5456-5463). Mouse mAbs to PspA have been shown to prolong survival of mice and improve efficacy of antibiotic treatment (Hikansson et al. 2001. Infect Immun 69:3372-3381). Additionally, antibodies isolated from humans following immunization with recombinant PspA protein are broadly cross-reactive and protect mice from pneumococcal infection with heterologous PspA (Nabors et al. (2000) Vaccine 18:1743-1754; and Briles et al. (2000) J Infect Dis 182:1694-1701). Mouse mAbs to PspA have been shown to prolong survival of mice, and improve efficacy of antibiotic treatment (Hikansson et al. 2001. Infect Immun 69:3372-3381). Additionally, antibodies isolated from humans following immunization with recombinant PspA are broadly cross-reactive and protect mice from pneumococcal infection with heterologous PspA (Nabors et al. (2000) Vaccine 18:1743-1754; and Briles et al. (2000) J Infect Dis 182:1694-1701). A clinical trial of a recombinant attenuated Salmonella typhi vaccine vector producing PspA has been completed (NCT01033409), and a protein-based Phase Ia clinical trial incorporating PspA is currently underway (NCT04087460).

Since PhtD and PspA are highly conserved across pneumococcal serotypes, mAbs to PhtD and PspA could prevent and/or treat disease from a broad-spectrum of pneumococcal serotypes.

Human mAbs are promising as therapeutics for bacterial pathogens, as bezlotoxumab was FDA approved for prevention of recurrent Clostridium difficile infection (Wilcox et al. (2017) N Engl J Med 376:305-317). However, there have been no human mAbs isolated to any pneumococcal protein antigens.

Human monoclonal antibodies to PhtD and PspA are disclosed herein. The serotype breadth, epitope specificity, and the protective efficacy of the disclosed antibodies was also demonstrated in multiple mouse models of pneumococcal infection.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as a PhtD or PspA. The term “antibody” is used herein in the broadest sense and encompasses various antibody structures, including but not limited to monoclonal antibodies, polyclonal antibodies, multi-specific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “V_{L}” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

Biological sample: A sample obtained from a subject. Biological samples include all clinical samples useful for detection of disease or infection in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having, suspected of having, or at risk of having a Streptococcus pneumoniae infection, or a subject having or suspected of having a viral respiratory infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody is linked to an effector molecule; for example, an antibody that specifically binds to Streptococcus pneumoniae covalently linked to an effector molecule, such as a detectable label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a Streptococcus pneumoniae-specific antibody can include up to 1, 2, 3, 4, 5, 6, 7, 8, 9, or up to 10 conservative substitutions compared to a reference antibody sequence and retain specific binding activity for PhtD or PspA. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to a PhtD or PspA. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not-infected by a Streptococcus pneumoniae. In other embodiments, the control is a positive control, such as a tissue sample obtained from a patient diagnosed with a Streptococcus pneumoniae infection. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to inhibit a Streptococcus pneumoniae infection, such as a Streptococcus pneumoniae serotype 3, serotype 4, or serotype 19A infection, to measurably alter outward symptoms of such an infection, or to reduce risk of getting a secondary infection.

In one example, a desired response is to inhibit, reduce, or prevent Streptococcus pneumoniae infection. The Streptococcus pneumoniae infection does not need to be completely eliminated or reduced or prevented for the method to be effective.

In some embodiments, administration of an effective amount of a disclosed antibody or antigen binding fragment can reduce or inhibit a Streptococcus pneumoniae infection (for example, as measured by titer, or by number or percentage of subjects infected by the Streptococcus pneumoniae or by an increase in the survival time of infected subjects, or reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody or antigen binding fragment that specifically binds the PhtD or PspA that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen load. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a Streptococcus pneumoniae surface protein, such as an epitope of PhtD or PspA.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to Kabat. For IgA, the Fc region includes immunoglobulin domains Cα2 and Cα3 and optionally the lower part of the hinge between Cα1 and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgG: A polypeptide belonging to the class or isotype of antibodies that are substantially encoded by a recognized immunoglobulin gamma gene. In humans, this class comprises IgG₁, IgG₂, IgG₃, and IgG4.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting or treating a disease: Inhibiting the full development of a disease or condition, for example, in a subject who is at risk for a disease such as a Streptococcus pneumoniae infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of bacterial infection, such as preventing or reducing the risk of Streptococcus pneumoniae infection. The beneficial effect can be evidenced, for example, by a delayed onset of clinical symptoms of the disease in a susceptible subject, a reduction in severity of some or all clinical symptoms of the disease, a slower progression of the disease, a reduction in the bacterial load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Pneumococcal disease: A disease caused by a Streptococcus pneumoniae infection. Invasive pneumococcal disease is a serious condition that often require hospitalization and can be fatal. Severe disease manifestations include pneumococcal pneumonia, sepsis, and/or meningitis. Common symptoms of pneumococcal pneumonia include fever, cough, shortness of breath, and chest pain. Symptoms of pneumococcal meningitis include stiff neck, fever, confusion and disorientation, and coma. Symptoms of pneumococcal sepsis are similar to the symptoms of pneumonia and meningitis, but include chills, a drop in blood pressure, and severe organ dysfunction. Certain chronic conditions can predispose a patient to develop invasive pneumococcal disease, such as diabetes, chronic obstructive pulmonary disease, cardiovascular diseases, and human immunodeficiency virus. Other risk factors include a viral respiratory tract infection, age, and a previous viral respiratory infection. Children under the age of 2 and adults over the age of 65 are more susceptible to pneumococcal disease.

Although vaccination is widespread in the developed world, pneumococcal infection remains responsible for 30% of adult pneumonia and has a mortality rate of 11-40%. (Bridy-Pappas et al. (2005) Pharmacotherapy 25:1193-212). Furthermore, in regions of the world with high childhood mortality rates, pneumococcal pneumonia is the cause of death for 20-50% of children (Williams et al. (2002) Lancet Infect Dis 2:25-32).

Pneumococcal histidine triad protein D (PhtD): PhtD is a member of a group of conserved surface proteins on S. pneumoniae that also includes PhtA, PhtB, and PhtC, all of which share histidine triad motifs (Adamou et al. (2001) Infect Immun 69:949-958). The proteins have high sequence homology to each other, and PhtB and PhtD share 87% sequence homology (Rioux et al. (2011) Microbiology 157:335-348). PhtD is highly conserved, varying 91-98% among strains isolated from invasive disease cases in children (Yun et al. (2015) PLoS One 10:e0134055). One study of 107 pneumococcal strains showed PhtD was expressed in 100% of tested serotypes, while other studies have found PhtD is widely prevalent but is absent in a subset of isolated strains (Rioux et al. (2011) Microbiology 157:335-348; Kawaguchiya et al. (2019) Pathog 8:162; Blumental et al. (2015) PLoS One 10:e0133885; and Rioux et al. (2011) Microbiology 157:336-348). The function of the Pht family of proteins has not been fully elucidated, although data has implicated the proteins in attachment of S. pneumoniae to respiratory epithelial cells (Plumptre et al. (2013) Infect Immun 2013/07/22. 81:3644-3651; Kallio et al. (2014) Infect Immun 2014/02/03. 82:1683-1691). In addition, the first histidine triad motif of PhtD has been shown to be important for zinc acquisition and bacterial homeostasis (Eijkelkamp et al. (2016) Infect Immun 84:407 LP—415). A crystal structure of the third histidine triad motif bound to Zn²⁺, and a solution NMR structure of the N-terminal fragment of PhtD has been determined (Luo et al. (2018) FEBS Lett 592:2341-2350). Exemplary amino acid and nucleic acid sequences for PhtD can be found in GENBANK® Accession No. AF318955.1, Jul. 14, 2016, incorporated herein by reference.

Pneumococcal surface protein A (PspA): Another vaccine antigen, PspA is an important virulence factor of S. pneumoniae and one of the most abundant surface proteins (Rosenow et al. (1997) Mol Microbiol 25:819-829). As with PhtD, PspA is found in the majority of examined clinical isolates (Hollingshead et al. (2006) J Med Microbiol 55:215-221). PspA mutant strains are cleared faster from the blood of mice compared to intact strains, and vaccination with PspA protects mice from pneumococcal infection (Briles et al. 1988. Rev Infect Dis 10 Suppl. 2:S372-4; Bosarge et al. (2001) Infect Immun 69:5456-5463). PspA is less conserved than PhtD, and is grouped into three families with >55% identity, and six clades with >75% identity (Hollingshead et al. (2000) Infect Immun 68:5889-5900). PspA has four distinct structural domains, including the alpha-helical region, the proline rich region, the choline binding repeat domain, and the cytoplasmic tail, of which the proline rich region is highly conserved across clades, while the N-terminal alpha-helical region is more variable (Khan and Jan (2017) Front Microbiol 8:742; Ren et al. (2004) Infect Immun 72:114-122; Mukerji et al. 2012. J Immunol 189:5327-5335; and Tu et al. (1999) Infect Immun 67:4720 LP-4724). PspA has been shown to inhibit complement deposition and has shown specificity for binding of human lactoferrin, although the importance of this binding is unclear. An X-ray crystal structure of the lactoferrin binding domain of PspA in complex with the N-terminal region of human lactoferrin has been determined (Senkovich et al. (2007) J Mol Biol 370:701-713). Exemplary amino acid and nucleic acid sequences for PspA can be found in GENBANK® Accession No. AF490268.1, Jul. 24, 2016, incorporated herein by reference.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Severe acute respiratory syndrome coronavirus (SARS-CoV)-2: Also known as Wuhan coronavirus or 2019 novel coronavirus, SARS-CoV-2 is a positive-sense, single stranded RNA virus of the genus betacoronavirus that has emerged as a highly fatal cause of severe acute respiratory infection. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994., Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example a PhtD or PspA, and does not bind in a significant amount to other proteins present in the sample or subject. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on a PhtD or PspA an antibody that binds substantially to the PhtD or PspA substrate to which the protein is attached, or the protein in a biological specimen. It is, of course, recognized that a certain degree of non-specific interaction may occur between an antibody and a non-target. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Streptococcus pneumoniae: A gram-positive, facultative anaerobic bacteria commonly found in the upper respiratory tract. Pneumococcal carriage precedes active infection. In young children, carriage rates of Streptococcus pneumoniae can be as high as 40-60%. Colonization is typically asymptomatic; however, Streptococcus pneumoniae can rapidly disseminate, often following a primary viral infection such as influenza, to cause pneumonia and invasive pneumococcal disease. Currently, there are at least 100 known serotypes, most of which can cause pneumococcal disease, including serotype 3, serotype 4, or serotype 19A.

Pneumonia is the most common disease caused S. pneumoniae. Symptoms include fever and chills, cough, rapid breathing, difficulty breathing, and chest pain. The elderly can also experience confusion, and low alertness. Streptococcus pneumoniae can also cause pneumococcal meningitis, which is an infection of the tissue covering the brain and spinal cord. Symptoms include stiff neck, fever, headache, confusion, and photophobia. Sepsis can also be caused by Streptococcus pneumoniae; sepsis can result in tissue damage, organ failure, and death. The symptoms include confusion, shortness of breath, elevated heart rate, pain or discomfort, over-perspiration, fever, shivering, or feeling cold.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a human. In an additional example, a subject is selected that is in need of inhibiting a Streptococcus pneumoniae infection. For example, the subject is either uninfected and at risk of the Streptococcus pneumoniae infection or is infected and in need of treatment.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some embodiments, a viral vector comprises a nucleic acid molecule encoding a disclosed antibody or antigen binding fragment that specifically binds to a Streptococcus pneumoniae. In some embodiments, the viral vector can be an adeno-associated virus (AAV) vector.

Under conditions sufficient for: A phrase that is used to describe any environment that permits a desired activity.

### II. Description of Several Embodiments

Isolated monoclonal antibodies and antigen binding fragments that specifically bind Streptococcus pneumoniae are provided. The antibodies and antigen binding fragments can be fully human. In some embodiments the disclosed antibodies can inhibit a Streptococcus pneumoniae infection in vivo, and can be administered prior to, or after, an infection with a Streptococcus pneumoniae, such as, but not limited to, Streptococcus pneumoniae serotype 3, serotype 4, or serotype 19A. Multi-specific antibodies (e.g., bispecific antibodies) including the variable domains of these antibodies are also provided. In addition, disclosed herein are compositions comprising the antibodies, multi-specific antibodies (e.g., bispecific antibodies), and antigen binding fragments and a pharmaceutically acceptable carrier. Nucleic acids encoding the antibodies, multi-specific antibodies (e.g., bispecific antibodies), antigen binding fragments, variable domains, and expression vectors (such as adeno-associated virus (AAV) viral vectors) comprising these nucleic acids are also provided. The antibodies, multi-specific antibodies (e.g., bispecific antibodies), antigen binding fragments, nucleic acid molecules, host cells, and compositions can be used for research, diagnostic, treatment and prophylactic purposes. For example, the disclosed antibodies and antigen binding fragments can be used to diagnose a subject with a Streptococcus pneumoniae infection or can be administered to inhibit a Streptococcus pneumoniae infection in a subject.

A. Monoclonal Antibodies that Specifically Bind a PhtD or PspA and Antigen Binding Fragments Thereof

The discussion of monoclonal antibodies below refers to isolated monoclonal antibodies that include heavy and/or light chain variable domains, or antigen binding fragments thereof, comprising a CDR1, CDR2, and/or CDR3 with reference to the IMGT numbering scheme (unless the context indicates otherwise). Various CDR numbering schemes (such as the Kabat, Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some embodiments, a monoclonal antibody is provided that comprises the heavy and light chain CDRs of any one of the antibodies described herein. In some embodiment, a monoclonal antibody is provided that comprises the heavy and light chain variable regions of any one of the antibodies described herein.

TABLE 1
IMGT CDRs of Antibodies and SEQ ID NOs
PhtD3 V_{H}
QVQLVQSGPDVKKPGSSVKVSCKASGAAFESFAFAWVRQAPGQGFEWMGRIIPILETRDYAEKFQGRMTMTT
DESTATAYMELNSLRFEDTAVYFCARDGHIMRTTLSDAALDVWGQGTTVIVSS (SEQ ID NO: 1)

| | SEQ ID NO: 1 | | CDR |
|---|---|---|---|
| V_{H} | positions | CDR protein sequence | SEQ ID NO |
| HCDR1 | 26-33 | GAAFESFA | 2 |
| HCDR2 | 51-58 | IIPILETR | 3 |
| HCDR3 | 97-114 | ARDGHIMRTTLSDAALDV | 4 |

PhtD3 V_{L}
DIVMTQSPVTLSLSPGERATLSCRASQSLTDNYLAWYQQKPGQAPRLLIYAASTRATGIPDRISGSGSGTDF
TLTISRVEPEDFAMFYCQQYQNSPFTFGGGTTVEIK (SEQ ID NO: 5)

| | SEQ ID NO: 5 | | CDR |
|---|---|---|---|
| V_{L} | positions | CDR protein sequence | SEQ ID NO |
| LCDR1 | 27-33 | QSLTDNY | 6 |
| LCDR2 | 51-53 | AAS | 7 |
| LCDR3 | 90-98 | QQYQNSPFT | 8 |

PhtD8 V_{H}
QVQLVQSGAEVKKPGASVKVSCKASGYTFTDYFIHWVRQAPGHGLEWMGWINPNRGVTNYTQKFQGRVTMTK
DTSVTSVYMELSRLTSDDTALYYCARGGTLDHWGQGTLVTVSS (SEQ ID NO: 9)

| | SEQ ID NO: 9 | | CDR |
|---|---|---|---|
| V_{H} | positions | CDR protein sequence | SEQ ID NO |
| HCDR1 | 26-33 | GYTFTDYF | 10 |
| HCDR2 | 51-58 | INPNRGVT | 11 |
| HCDR3 | 97-104 | ARGGTLDH | 12 |

PhtD8 V_{L}
QLVLTQSPSASASLGASVTLTCTLSSGHSTYDIAWHQQQPGKGPRHLMRLNGDGSHTNGDGIPDRFSGSSSG
AERYLTISSLQSEDEADYYCHTWVTNIHLVFGGGTKLTVL (SEQ ID NO: 13)

| | SEQ ID NO: 13 | | CDR |
|---|---|---|---|
| V_{L} | positions | CDR protein sequence | SEQ ID NO |
| LCDR1 | 26-32 | SGHSTYD | 14 |
| LCDR2 | 50-56 | LNGDGSH | 15 |
| LCDR3 | 93-102 | HTWVTNIHLV | 16 |

PhtD6 V_{H}
QVQLVQSGTEVKKPGASVKVACKASGYTFTSYDINWVRQAPGQGLEWMGWMNANSGNTGYAQKFQGRVTMTR
NTSITTAYMDLIDLTSEDTAIYYCARGPYWVENWFDTWGQGTLVSVSS (SEQ ID NO: 17)

| | SEQ ID NO: 17 | | CDR |
|---|---|---|---|
| V_{H} | positions | CDR protein sequence | SEQ ID NO |
| HCDR1 | 26-33 | GYTFTSYD | 18 |
| HCDR2 | 51-58 | MNANSGNT | 19 |
| HCDR3 | 97-109 | ARGPYWVENWFDT | 20 |

PhtD6 V_{L}
DIQMTQSPSSLSASVGDRVTITCRASRSIRSFLNWYQQKPGKPPNLLIYKASTLHSGVPSRFSGSGSGTDFT
LTINNLQPEDFATYYCQQSYSNQKTFGQGTKVDIK (SEQ ID NO: 21)

| | SEQ ID NO: 21 | | CDR |
|---|---|---|---|
| V_{L} | positions | CDR protein sequence | SEQ ID NO |
| LCDR1 | 27-32 | RSIRSF | 22 |
| LCDR2 | 50-52 | KAS | 23 |
| LCDR3 | 89-97 | QQSYSNQKT | 24 |

PhtD7 V_{H}
EVQLVQSGAEVKKPGASVKVSCKASGDIFSDSYIHWVRQAPGQGPEWMGWVSPNTGATHYAQKLQGRVTMTS
DTSISTAYLELTRLASDDTAVYYCARVLRGSYDFRGNYPHDFDYWGQGTLVTVSS (SEQ ID NO: 25)

| | SEQ ID NO: 25 | | CDR |
|---|---|---|---|
| V_{H} | positions | CDR protein sequence | SEQ ID NO |
| HCDR1 | 26-33 | GDIFSDSY | 26 |
| HCDR2 | 51-58 | VSPNTGAT | 27 |
| HCDR3 | 97-116 | ARVLRGSYDFRGNYPHDFDY | 28 |

PhtD7 V_{L}
QLVLTQPPSASASLGASVTLTCTLSRGHNNYPIAWLQKQTDKGPRYVMRLNSDGSHHKGDGIPDRFSGSSSG
AERYLSISSLQPEDEAEYYCQTWDTGLQGVFGGGTKLFVL (SEQ ID NO: 29)

| | SEQ ID NO: 29 | | CDR |
|---|---|---|---|
| V_{L} | positions | CDR protein sequence | SEQ ID NO |
| LCDR1 | 26-32 | RGHNNYP | 30 |
| LCDR2 | 50-56 | LNSDGSH | 31 |
| LCDR3 | 93-102 | QTWDTGLQGV | 32 |

PspA16 V_{H}
QVQLVQSGPDVKKPGASVKVSCKTSGYTFTGYYMHWVRQAPGQGLEWMGWVNPNTGGTSYAQKFQGRVTVTR
DTSISTVYMELSALGSDDTAIYFCARAWAPGAEYLHHWGQGTLVTVSS (SEQ ID NO: 33)

| | SEQ ID NO: 33 | | CDR |
|---|---|---|---|
| VH | positions | CDR protein sequence | SEQ ID NO |
| HCDR1 | 26-33 | GYTFTGYY | 34 |
| HCDR2 | 51-58 | VNPNTGGT | 35 |
| HCDR3 | 97-109 | ARAWAPGAEYLHH | 36 |

PspA16 V_{L}
EIVMTQSPGTLSLSPGETATLSCRASQSVGSSYLAWYQQKPGQAPRLLIFGASNRATGIPVRFSASGSGTDF
TLTISRLEPEDFAVYYCQQHDHSPFTFGPGTKVDLK (SEQ ID NO: 37)

| | SEQ ID NO: 37 | | CDR |
|---|---|---|---|
| V_{L} | positions | CDR protein sequence | SEQ ID NO |
| LCDR1 | 27-33 | QSVGSSY | 38 |
| LCDR2 | 51-53 | GAS | 39 |
| LCDR3 | 90-98 | QQHDHSPFT | 40 |

a. Monoclonal Antibody

In some embodiments, the antibody or antigen binding fragment is based on or is derived from the PhtD3 antibody and specifically binds to Streptococcus pneumoniae.

Monoclonal antibody PhtD3 targets the N-terminal portion of PhtD, a conserved surface protein on S. pneumoniae. As discussed further in the Examples, PhtD3 mAbs prolonged the survival of mice infected with pneumococcal serotype 3, and provide prophylactic protection against serotype 4 in both intranasal and intravenous infection models. Furthermore, mAb PhtD3 was protective in a serotype 3 treatment model when administered 24 hours after pneumococcal infection.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the PhtD3 antibody, and specifically binds to Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a Streptococcus pneumoniae. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a Streptococcus pneumoniae. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to a Streptococcus pneumoniae. In further examples, the disclosed antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 7, and 8, respectively, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 7, and 8, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigen binding fragment specifically binds to a Streptococcus pneumoniae. In some examples, the antibody or antigen binding fragment specifically binds a Streptococcus pneumoniae surface protein, PhtD. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a Streptococcus pneumoniae. In some examples, the antibody or antigen binding fragment specifically binds a Streptococcus pneumoniae surface protein, PhtD. In more embodiments, the antibody or antigen binding fragment comprises a V_{L} comprising the amino acid sequence set forth as SEQ ID NO: 5, and specifically binds to a Streptococcus pneumoniae. In some examples, the antibody or antigen binding fragment specifically binds a Streptococcus pneumoniae surface protein, PhtD. In some embodiments, the antibody or antigen binding fragment comprises a V_{H} and a V_{L} Comprising the amino acid sequences set forth as SEQ ID NOs: 1 and 5, respectively, and specifically binds to a Streptococcus pneumoniae. In some examples, the antibody or antigen binding fragment specifically binds a Streptococcus pneumoniae surface protein, PhtD.

In some embodiments, the disclosed antibodies inhibit Streptococcus pneumoniae infection.

b. Monoclonal Antibody PhtD8

In some embodiments, the antibody or antigen binding fragment is based on or is derived from the PhtD8 antibody and specifically binds to a Streptococcus pneumoniae.

Monoclonal antibody PhtD8 targets the C-terminal portion of PhtD, a conserved surface protein on Streptococcus pneumoniae. As discussed further in the Examples, PhtD8 is broadly reactive across multiple unrelated pneumococcal serotypes, and PhtD8 mAbs prolonged the survival of mice infected with pneumococcal serotype 3.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the PhtD8 antibody, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to a Streptococcus pneumoniae. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to a Streptococcus pneumoniae. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 13, respectively, and specifically binds to a Streptococcus pneumoniae. In further examples, the disclosed antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 10, 11, and 12, respectively, and/or a V_{L} comprising a LCDR1, a LCDR2, and a LCDR3 as set forth as SEQ ID NOs: 14, 15, and 16, respectively, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 10, 11, and 12, respectively, a V_{L} comprising a LCDR1, a LCDR2, and a LCDR3 as set forth as SEQ ID NOs: 14, 15, and 16, respectively, wherein the V_{H} comprises an amino acid sequence at least 90% identical to SEQ ID NO: 9, such as 95%, 96%, 97%, 98% or 99% identical to SEQ ID NO: 9, and wherein the V_{L} comprises an amino acid sequence at least 90% identical to SEQ ID NO: 13, such as 95%, 96%, 97%, 98% or 99% identical to SEQ ID NO: 13, and the antibody or antigens binding fragment specifically binds to a Streptococcus pneumoniae. In some examples, the antibody or antigen binding fragment specifically binds a Streptococcus pneumoniae surface protein, PhtD. In this embodiment, variations due to sequence identify fall outside the CDRs.

In some embodiments, the antibody or antigen binding fragment comprises a V_{H} comprising the amino acid sequence set forth as SEQ ID NO: 9, and specifically binds to a Streptococcus pneumoniae. In more embodiments, the antibody or antigen binding fragment comprises a V_{L} comprising the amino acid sequence set forth as SEQ ID NO: 13, and specifically binds to a Streptococcus pneumoniae. In some embodiments, the antibody or antigen binding fragment comprises a V_{H} and a V_{L} comprising the amino acid sequences set forth as SEQ ID NOs: 9 and 13, respectively, and specifically binds to a Streptococcus pneumoniae. In some examples, the disclosed antibody or antigen binding fragment specifically binds a Streptococcus pneumoniae surface protein, PhtD.

In some embodiments, the disclosed antibodies inhibit Streptococcus pneumoniae infection.

c. Monoclonal Antibody PhtD6

In some embodiments, the antibody or antigen binding fragment is based on or derived from the PhtD6 antibody, and specifically binds to a Streptococcus pneumoniae.

Monoclonal antibody PhtD6 targets the N-terminal portion of PhtD, a conserved surface protein on Streptococcus pneumoniae. As discussed further in the Examples, PhtD6 is broadly reactive across multiple unrelated pneumococcal serotypes, and has higher avidity to fixed bacteria than PhtD8. PhtD6 also shows intermediate binding competition with PhtD3. It is a striking observation that the N-terminal specificity of mAbs PhtD3 and PhtD6 correlates with higher binding to whole cell bacteria as compared to PhtD8 since the N-terminal region of the protein is predicted to be attached to the bacterial surface, leaving the C-terminal half more surface exposed.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the PhtD6 antibody, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to a Streptococcus pneumoniae. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 21, and specifically binds to a Streptococcus pneumoniae. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 21, respectively, and specifically binds to a Streptococcus pneumoniae. In further examples, the disclosed antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 18, 19, and 20 respectively, and/or a V_{L} comprising a LCDR1, a LCDR2, and a LCDR3 as set forth as SEQ ID NOs: 22, 23, and 24, respectively, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 18, 19, and 20, respectively, a V_{L} comprising a LCDR1, a LCDR2, and a LCDR3 as set forth as SEQ ID NOs: 22, 23, and 24, respectively, wherein the V_{H} comprises an amino acid sequence at least 90% identical to SEQ ID NO: 17, such as 95%, 96%, 97%, 98% or 99% identical to SEQ ID NO: 17, and wherein the V_{L} comprises an amino acid sequence at least 90% identical to SEQ ID NO: 21, such as 95%, 96%, 97%, 98% or 99% identical to SEQ ID NO: 21, and the antibody or antigens binding fragment specifically binds to a Streptococcus pneumoniae. In some examples, the antibody or antigen binding fragment specifically binds a Streptococcus pneumoniae surface protein, PhtD. In this embodiment, variations due to sequence identify fall outside the CDRs.

In some embodiments, the antibody or antigen binding fragment comprises a V_{H} comprising the amino acid sequence set forth as SEQ ID NO: 17, and specifically binds to a Streptococcus pneumoniae. In more embodiments, the antibody or antigen binding fragment comprises a V_{L} comprising the amino acid sequence set forth as SEQ ID NO: 21, and specifically binds to a Streptococcus pneumoniae. In some embodiments, the antibody or antigen binding fragment comprises a V_{H} and a V_{L} comprising the amino acid sequences set forth as SEQ ID NOs: 17 and 21, respectively, and specifically binds to a Streptococcus pneumoniae. In some examples, the disclosed antibody or antigen binding fragment specifically binds a Streptococcus pneumoniae surface protein, PhtD.

In some embodiments, the disclosed antibodies inhibit Streptococcus pneumoniae infection.

d. Monoclonal Antibody PhtD7

In some embodiments, the antibody or antigen binding fragment is based on or is derived from the PhtD7 antibody and specifically binds to a Streptococcus pneumoniae.

Monoclonal antibody PhtD7 appears to target a unique conformational epitope that is dependent on amino acids 341-838, but this mAb does not bind 341-647 or 645-838 fragments of PhtD. As discussed further in the Examples, PhtD7 is broadly reactive across multiple unrelated pneumococcal serotypes, and has higher avidity to fixed bacteria than PhtD8. Furthermore, PhtD7 mAbs were protective and increased survival in a mouse model of a Streptococcus pneumoniae infection.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the PhtD7 antibody, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 25, and specifically binds to a Streptococcus pneumoniae. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 29, and specifically binds to a Streptococcus pneumoniae. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 25 and 29, respectively, and specifically binds to a Streptococcus pneumoniae. In further examples, the disclosed antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 26, 27, and 28 respectively, and/or a V_{L} comprising a LCDR1, a LCDR2, and a LCDR3 as set forth as SEQ ID NOs: 30, 31, and 32, respectively, and specifically binds to a Streptococcus pneumoniae. In some examples, the antibody or antigen binding fragment specifically binds a Streptococcus pneumoniae surface protein, PhtD.

In some embodiments, the antibody or antigen binding fragment comprises a V_{H} comprising a HCDR1, a HCDR2, and a HCDR3 as set forth as SEQ ID NOs: 26, 27, and 28, respectively, a V_{L} comprising a LCDR1, a LCDR2, and a LCDR3 as set forth as SEQ ID NOs: 30, 31, and 32, respectively, wherein the V_{H} comprises an amino acid sequence at least 90% identical to SEQ ID NO: 25, such as 95%, 96%, 97%, 98% or 99% identical to SEQ ID NO: 25, and wherein the V_{L} comprises an amino acid sequence at least 90% identical to SEQ ID NO: 29, such as 95%, 96%, 97%, 98% or 99% identical to SEQ ID NO: 29, and the antibody or antigens binding fragment specifically binds to a Streptococcus pneumoniae. In some examples, the antibody or antigen binding fragment specifically binds a Streptococcus pneumoniae surface protein, PhtD. In this embodiment, variations due to sequence identify fall outside the CDRs.

In some embodiments, the antibody or antigen binding fragment comprises a V_{H} comprising the amino acid sequence set forth as SEQ ID NO: 25, and specifically binds to a Streptococcus pneumoniae. In more embodiments, the antibody or antigen binding fragment comprises a V_{L}comprising the amino acid sequence set forth as SEQ ID NO: 29, and specifically binds to a Streptococcus pneumoniae. In some embodiments, the antibody or antigen binding fragment comprises a V_{H} and a V_{L} comprising the amino acid sequences set forth as SEQ ID NOs: 25 and 29, respectively, and specifically binds to a Streptococcus pneumoniae. In some examples, the disclosed antibody or antigen binding fragment specifically binds a Streptococcus pneumoniae surface protein, PhtD.

In some embodiments, the disclosed antibodies inhibit Streptococcus pneumoniae infection.

e. Monoclonal Antibody PspA16

In some embodiments, the antibody or antigen binding fragment is based on or is derived from the PhtD8 antibody and specifically binds to a Streptococcus pneumoniae.

Monoclonal antibody PspA16 targets the N-terminal segment of PspA, one of the most abundant surface protein on Streptococcus pneumoniae. As discussed further in the Examples, PspA16 has high avidity to recombinant PspA, and binds to the N-terminal fragment 1-247 based on positive binding to amino acid fragments 1-438 and 1-512, and negative binding to 247-512, 436-725, and 247-725 fragments. PspA16 was found to bind Streptococcus pneumoniae serotype 19A and serotype strain 3 WU. Since PspA16 binds to the most variable region of PspA, the reduced binding to divergent serotypes was expected. Furthermore, as shown in the Examples, PspA16 mAbs were protective and increased survival in a mouse model of a Streptococcus pneumoniae infection.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the PspA16 antibody, and specifically binds to a Streptococcus pneumoniae. In some examples, the antibody or antigen binding fragment specifically binds a Streptococcus pneumoniae surface protein, PspA.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 33, and specifically binds to a Streptococcus pneumoniae. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 37, and specifically binds to a Streptococcus pneumoniae. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 33 and 37, respectively, and specifically binds to a Streptococcus pneumoniae. In some examples, the disclosed antibody or antigen binding fragment specifically binds a Streptococcus pneumoniae surface protein, PspA.

In some embodiments, the antibody or antigen binding fragment comprises a V_{H} comprising a HCDR1, a HCDR2, and a HCDR3 as set forth as SEQ ID NOs: 34, 35, and 36, respectively, and/or a V_{L} comprising a LCDR1, a LCDR2, and a LCDR3 as set forth as SEQ ID NOs: 38, 39, and 40, respectively, and specifically binds to a Streptococcus pneumoniae. In some examples, the disclosed antibody or antigen binding fragment specifically binds a Streptococcus pneumoniae surface protein, PspA.

In some embodiments, the antibody or antigen binding fragment comprises a V_{H} comprising a HCDR1, a HCDR2, and a HCDR3 as set forth as SEQ ID NOs: 34, 35, and 36, respectively, a V_{L} comprising a LCDR1, a LCDR2, and a LCDR3 as set forth as SEQ ID NOs: 38, 39, and 40, respectively, wherein the V_{H} comprises an amino acid sequence at least 90% identical to SEQ ID NO: 33, such as 95%, 96%, 97%, 98% or 99% identical to SEQ ID NO: 33, and wherein the V_{L} comprises an amino acid sequence at least 90% identical to SEQ ID NO: 37, such as 95%, 96%, 97%, 98% or 99% identical to SEQ ID NO: 37, and the antibody or antigens binding fragment specifically binds to a Streptococcus pneumoniae. In some examples, the disclosed antibody or antigen binding fragment specifically binds a Streptococcus pneumoniae surface protein, PspA. In this embodiment, variations due to sequence identify fall outside the CDRs.

In some embodiments, the antibody or antigen binding fragment comprises a V_{H} comprising the amino acid sequence set forth as SEQ ID NO: 33, and specifically binds to a Streptococcus pneumoniae. In more embodiments, the antibody or antigen binding fragment comprises a V_{L} comprising the amino acid sequence set forth as SEQ ID NO: 37, and specifically binds to a Streptococcus pneumoniae. In some embodiments, the antibody or antigen binding fragment comprises a V_{H} and a V_{L} comprising the amino acid sequences set forth as SEQ ID NOs: 33 and 37, respectively, and specifically binds to a Streptococcus pneumoniae. In some examples, the disclosed antibody or antigen binding fragment specifically binds a Streptococcus pneumoniae surface protein, PspA.

In some embodiments, the disclosed antibodies inhibit Streptococcus pneumoniae infection.

f. Additional Antibodies

In some examples, antibodies that bind to an epitope of interest can be identified based on their ability to cross-compete (for example, to competitively inhibit the binding of, in a statistically significant manner) with the antibodies provided herein in binding assays. In other examples, antibodies that bind to an epitope of interest can be identified based on their ability to cross-compete (for example, to competitively inhibit the binding of, in a statistically significant manner) with the one or more of the antibodies provided herein in binding assays.

Human antibodies that bind to the same epitope of the PhtD or PspA to which the disclosed antibodies bind can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

### 2. Additional Description of Antibodies and Antigen Binding Fragments

An antibody or antigen binding fragment of the antibodies disclosed herein can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgA, IgM or an IgG antibody, such as IgG₁, IgG₂, IgG₃, or IgG₄. The class of an antibody that specifically binds to a Streptococcus pneumoniae can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or CH from a different class of immunoglobulin molecule. This can be achieved, for example, using a vector or nucleic acid molecule that comprises a C_{L} or CH chain. For example, an antibody that specifically binds Streptococcus pneumoniae, that was originally IgG may be class switched to an IgA. Class switching can be used to convert one IgG subclass to another, such as from IgG₁ to IgG2, IgG3, or IgG4.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to the Streptococcus pneumoniae, such as to PhtD or PspA, is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several embodiments, the antibody or antigen binding fragment specifically binds Streptococcus pneumoniae, such as to PhtD or PspA, with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20@) in PBS. When the plates have dried, 150 μl/well of scintillant (MICROSCINT™-20; PerkinElmer) is added, and the plates are counted on a TOPCOUNT™ gamma counter (PerkinElmer) for ten minutes. Concentrations of each Fab that give less than or equal to 20% of maximal binding are chosen for use in competitive binding assays.

In another assay, K_{D} can be measured using surface plasmon resonance assays using Biolayer interferometry (BLI), see the examples section. In other embodiments, KD can be measured using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is Calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Affinity can also be measured by high throughput SPR using the Carterra LSA.

(b) Multi-Specific Antibodies

In some embodiments, a multi-specific antibody, such as a bispecific antibody, is provided that comprises an antibody or antigen binding fragment that specifically binds Streptococcus pneumoniae, such as to PhtD or PspA, as provided herein. Any suitable method can be used to design and produce the multi-specific antibody, such as crosslinking two or more antibodies (e.g. crosslinking an antibody that specifically binds PhtD and an antibody that specifically binds PspA), antigen binding fragments (such as scFvs) of the same type or of different types. Exemplary methods of making multi-specific antibodies include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

In some embodiments, the multispecific antibody includes a PhtD3 antibody or antigen binding fragment. In further embodiments, the multispecific antibody includes a PhtD3 antibody or antigen binding fragment and one or more of a PhtD7, PhtD6, PhtD8, and PspA16 antibody or antigen binding fragment. In one example, the multispecific antibody includes a PhtD3 antibody or antigen binding fragment and a PhtD7 antibody or antigen binding fragment. In another example, the multispecific antibody includes a PhtD3 antibody or antigen binding fragment and a PhtD6 antibody or antigen binding fragment. In a further example, the multispecific antibody includes a PhtD3 antibody or antigen binding fragment and a PhtD8 antibody or antigen binding fragment. In some examples, the multispecific antibody includes a PhtD3 antibody or antigen binding fragment and a PspA16 antibody or antigen binding fragment.

In some embodiments, the multispecific antibody includes a PhtD7 antibody or antigen binding fragment. In further embodiments, the multispecific antibody includes a PhtD7 antibody or antigen binding fragment and one or more of a PhtD3, PhtD6, PhtD8, and PspA16 antibody or antigen binding fragment. In one example, the multispecific antibody includes a PhtD7 antibody or antigen binding fragment and a PhtD6 antibody or antigen binding fragment. In a further example, the multispecific antibody includes a PhtD7 antibody or antigen binding fragment and a PhtD8 antibody or antigen binding fragment. In some examples, the multispecific antibody includes a PhtD7 antibody or antigen binding fragment and a PspA16 antibody or antigen binding fragment.

In some embodiments, the multispecific antibody includes a PhtD6 antibody or antigen binding fragment. In further embodiments, the multispecific antibody includes a PhtD6 antibody or antigen binding fragment and one or more of a PhtD3, PhtD7, PhtD8, and PspA16 antibody or antigen binding fragment. In one example, the multispecific antibody includes a PhtD6 antibody or antigen binding fragment and a PhtD8 antibody or antigen binding fragment. In a further example, the multispecific antibody includes a PhtD6 antibody or antigen binding fragment and a PspA16 antibody or antigen binding fragment.

In some embodiments, the multispecific antibody includes a PhtD8 antibody or antigen binding fragment. In further embodiments, the multispecific antibody includes a PhtD8 antibody or antigen binding fragment and one or more of a PhtD3, PhtD7, PhtD6, and PspA16 antibody or antigen binding fragment. In one example, the multispecific antibody includes a PhtD8 and a PspA16 antibody or antigen binding fragment. In some embodiments, the multispecific antibody includes a PspA16 antibody or antigen binding fragment. In further embodiments, the multispecific antibody includes a PspA16 antibody or antigen binding fragment and one or more of a PhtD3, PhtD7, PhtD6, and PhtD8 antibody or antigen binding fragment.

The multi-specific antibody may have any suitable format that allows for binding to Streptococcus pneumoniae, such as to PhtD or PspA, by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Brühl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

The outermost or N-terminal variable domain is termed VD1 and the innermost variable domain is termed VD2; the VD2 is proximal to the C-terminal CH1 or CL. As disclosed in Jakob et al., supra, DVD-immunoglobulin molecules can be manufactured and purified to homogeneity in large quantities, have pharmacological properties similar to those of a conventional IgG₁, and show in vivo efficacy. Any of the disclosed monoclonal antibodies can be included in a DVD-immunoglobulin format.

### (c) Antigen Binding Fragments

Antigen binding fragments are encompassed by the present disclosure, such as Fab, F(ab′)₂, and Fv which include a heavy chain and V_{L} and specifically bind Streptococcus pneumoniae, such as to PhtD or PspA. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some embodiments, amino acid sequence variants of the antibodies and multi-specific antibodies (e.g., bispecific antibodies) provided herein are provided. For example, it may be desirable to improve the binding affinity and/or other biological properties of the antibody or multispecific antibody. Amino acid sequence variants of an antibody may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In further embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21.

In yet other embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 25. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29.

In more embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 33. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 37.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant V_{H} and V_{L} sequences provided above, each CDR either is unaltered, or contains no more than one, two or three amino acid substitutions. In some embodiments of the variant V_{H} and V_{L} sequences provided above, only the framework residues are modified so the CDRs are unchanged.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for Streptococcus pneumoniae, such determining binding affinity for PhtD or PspA. In particular examples, the V_{H} amino acid sequence is one of SEQ ID NOs: 1, 9, 17, 25, or 33. In other examples, the V_{L} amino acid sequence is one of SEQ ID NOs: 5, 13, 21, 29, or 37, respectively.

In some embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lec 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several embodiments, the constant region of the antibody or multispecific antibody comprises one or more amino acid substitutions to optimize in vivo half-life of the antibody. The serum half-life of IgG Abs is regulated by the neonatal Fc receptor (FcRn). Thus, in several embodiments, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise an Fc polypeptide including any of the substitutions listed above, for example, the Fc polypeptide can include the M428L and N434S substitutions.

In some embodiments, an antibody or multi-specific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies, antigen binding fragments, and multi-specific antibodies (e.g., bispecific antibodies) that specifically bind to Streptococcus pneumoniae, such as to PhtD or PspA, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵, ³²P, ¹⁴C, ³H and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody, antigen binding fragment, or multi-specific antibody varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody, antigen binding fragment, or multi-specific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or multi-specific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or multi-specific antibody (e.g., bispecific antibody) can be determined.

The antibody, antigen binding fragment or multi-specific antibody (e.g., bispecific antibody) can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or multi-specific antibody, can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or multi-specific antibody, may also be conjugated with biotin, and detected through indirect measurement of avidin or streptavidin binding. It should be noted that the avidin itself can be conjugated with an enzyme or a fluorescent label.

The antibody, antigen binding fragment or multi-specific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or multi-specific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or multi-specific antibody, can also be conjugated with a radiolabeled amino acid, for example, for diagnostic purposes. For instance, the radiolabel may be used to detect Streptococcus pneumoniae by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or multi-specific antibody in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules, such as mRNA) encoding the amino acid sequences of antibodies, antigen binding fragments, multi-specific antibodies (e.g., bispecific antibodies), and conjugates that specifically bind to Streptococcus pneumoniae, such as to PhtD or PspA, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several embodiments, nucleic acid molecules can encode the V_{H}, the V_{L}, or both the V_{H} and V_{L} (for example in a bicistronic expression vector) of a disclosed antibody or antigen binding fragment. In some embodiments, the nucleic acid molecules encode an scFv. In several embodiments, the nucleic acid molecules can be expressed in a host cell (such as a mammalian cell) to produce a disclosed antibody or antigen binding fragment. Nucleic acid molecules encoding an scFv are provided.

The genetic code can be used to construct a variety of functionally equivalent nucleic acid sequences, such as nucleic acids which differ in sequence but which encode the same antibody sequence, or encode a conjugate or fusion protein including the V_{L} and/or V_{H} nucleic acid sequence.

In a non-limiting example, an isolated nucleic acid molecule encodes the V_{H} of the PhtD3, PhtD6, PhtD7, PhtD8, or PspA16 antibody. Exemplary nucleic acid sequences are provided herein. In another non-limiting example, the nucleic acid molecule encodes the V_{L} of the PhtD3, PhtD6, PhtD7, PhtD8, or PspA16 antibody. In further non-limiting examples, the nucleic acid molecule can encode a bi-specific antibody, such as in DVD-immunoglobulin format. The nucleic acid can also encode an scFv. The nucleic acid molecule can also encode a conjugate.

Exemplary nucleotide sequences are disclosed below:

PhtD3 V_{H}
(SEQ ID NO: 41)
caggtgcagctagtgcagtctgggcctgacgtgaagaagcctgggtcc
tcggtgaaggtctcctgcaaggcctctggagccgccttcgagagtttt
gccttcgcctgggtgcgacaggcccctggacaagggtttgagtggatg
ggaaggatcattccaatcttggaaacaagggactacgcagagaagttc
cagggcagaatgacgatgaccacagacgagtcgacggcgacagcctac
atggaactgaacagcctaagatttgaagacacggccgtttatttctgt
gcgcgagatgggcacattatgaggacaactctctcggatgctgcactt
aggatgtctggggccaagggacaacggtcattgtctcctc
PhtD3 V_{L}
(SEQ ID NO: 42)
gacatcgtgatgacccagtctccagtcaccctgtctttgtctccaggg
gagagagccaccctctcctgcagggccagtcagagtcttactgacaac
tacttagcctggtaccagcagaaacctggccaggctcccaggctcctc
atctacgccgcatccaccagggccactggcatcccagacaggatcagt
ggcagtgggtctgggacagacttcactctcaccatcagcagagtggag
cctgaagattttgcaatgttttactgtcaacagtatcagaactcaccg
ttcaccttcggggggggaccacggtggagatcaaac
PhtD8 V_{H}
(SEQ ID NO: 43)
caggtgcagctggtgcagtctggggctgaggtgaagaagcctggggcc
tcagtgaaggtctcctgtaaggcttctggatacaccttcaccgactac
tttatacactgggtgcgacaggcccctggacacggtcttgaatggatg
gggtggatcaaccctaaccgcggtgtcacaaactatacacagaagttt
cagggcagggtcaccatgaccaaggacacgtccgtcacctcagtctac
atggagctgagcaggctgacatctgacgacacggccctatattattgt
gcgagaggtggtacgcttgaccactggggccagggcaccctggtcacc
gtctcctctg
PhtD8 V_{L}
(SEQ ID NO: 44)
cagcttgtgctgactcaatcgccctctgcctctgcctccctgggagcc
tcggtcaccctcacctgcactctgagcagtgggcacagcacctacgac
atcgcatggcatcagcagcagccaggaaagggccctcgacacttgatg
agacttaacggtgatggcagtcacaccaacggggacgggatccctgat
cgcttctcaggctccagctctggggctgagcgctacctcaccatctcc
agcctccagtctgaagatgaggctgactattactgtcacacctgggtc
actaacattcatttggtgttcggcggagggaccaaactgaccgtccta
g
PhtD6 V_{H}
(SEQ ID NO: 45)
caggtgcagttggtgcagtctgggactgaggtgaagaagcctggggcc
tcagtgaaggtcgcctgcaaggcttctggatacaccttcactagttat
gatatcaactgggtgcgacaggcccctggacaagggcttgagtggatg
ggatggatgaacgcgaacagtggcaacacaggctatgcacaaaagttc
cagggcagagtcaccatgaccaggaacacctccattaccacagcctac
atggacctgattgatctgacatctgaggacacggccatatattactgt
gcgagagggccgtactgggtggagaattggttcgacacctggggccag
ggaaccctggtcagcgtctcctcag
PhtD6 V_{L}
(SEQ ID NO: 46)
gacatccagatgacccagtctccatcctccctgtctgcgtctgtcgga
gacagagtcaccatcacttgccgggcaagtcggagcattcgcagcttt
ttaaattggtatcaacaaaaaccagggaaaccccctaacctcctgatc
tataaagcatccactttgcacagtggggtcccgtctaggttcagtggc
agtggatctgggacagatttcactctcacaatcaacaatctacaaccc
gaagattttgcaacttactactgtcaacagagttacagtaatcagaag
accttcggccaagggaccaaggtggacatcaaac
PhtD7 V_{H}
(SEQ ID NO: 47)
gaggtgcagctggtgcagtctggggctgaagtgaagaagcctggggcc
tcagtgaaggtctcctgcaaggcttctggagacatcttcagcgactcc
tatattcactgggtgcgacaggcccctggacaagggcctgagtggatg
ggatgggtcagccctaacactggtgccacacattatgcacagaagttg
cagggcagagtcaccatgaccagcgacacgtccatcagtacagcctat
ttggagctgaccaggctggcatctgacgacacggccgtttattactgt
gcgagagtcttaaggggaagttatgatttccggggtaattatccacat
ctcctcaggattttgactactggggccagggaaccctggtcaccgt
PhtD7 V_{L}
(SEQ ID NO: 48)
cagcttgtgctgactcaaccgccctctgcctctgcctccctgggagcc
tcggtcaccctcacctgcactctgagcagaggacacaacaactacccc
atcgcttggctccaaaagcagacagataagggccctcgttatgtgatg
agacttaatagtgatggcagccaccacaagggggacggaatccctgat
cgcttctcaggctccagttctggggctgagcgctacctcagcatttcc
agtctccagcctgaagatgaggctgaatactactgtcagacgtgggac
actggccttcagggggtgttcggcggagggaccaaactgttcgtccta
g
PspA16 V_{H}
(SEQ ID NO: 49)
caggtgcagctggtgcagtctgggcctgacgtgaagaagcctggggcc
tcagtgaaggtctcctgtaagacttctggatacaccttcactggctac
tatatgcactgggtgcgacaggcccctggacaagggcttgagtggatg
ggatgggtcaaccctaacaccggtggcacaagttatgcacagaagttt
cagggcagggtcaccgtgaccagggacacgtccatcagcacagtctac
atggaactgagcgctctaggatctgacgacacggccatatatttctgt
gcgagggcgtgggctccgggcgctgagtacctccaccactggggccag
ggcaccctggtcaccgtctcctcag
PspA16 V_{L}
(SEQ ID NO: 50)
gagattgtgatgacccagtctccaggcaccctgtctttgtctccaggg
gaaacagccaccctctcctgcagggccagtcagagtgttggcagcagc
tacttagcctggtatcagcagaaacctggccaggctcccaggctcctc
atctttggtgcgtccaacagggccactggcatcccagtcaggttcagt
gccagtgggtctgggacagacttcactctcaccatcagcagactggag
cctgaagatttcgcagtgtattactgtcagcagcatgatcactcacca
ttcactttcggccctgggaccaaagtggatctcaaac

Degenerate variants of these nucleic acid sequence are also provided.

Nucleic acid molecules encoding the antibodies, antigen binding fragments, multi-specific antibodies (e.g., bispecific antibodies), and conjugates that specifically bind to Streptococcus pneumoniae, such as to PhtD or PspA, can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and Dubel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Multi-specific or polyvalent antibodies may be generated that bind specifically to a Streptococcus pneumoniae, such as to PhtD, and/or PspA, and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-Ig™ format.

One or more DNA sequences encoding the antibodies, antigen binding fragments, multi-specific antibodies (e.g., bispecific antibodies), or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies, antigen binding fragments, and multi-specific antibodies described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, multi-specific antibodies, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylactically, the polypeptides should be substantially free of endotoxin.

Methods for expression of antibodies, antigen binding fragments, multi-specific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

1. Inhibiting a Streptococcus pneumoniae Infection and/or Pneumococcal Disease

Methods are disclosed herein for the inhibition of a Streptococcus pneumoniae infection in a subject. The Streptococcus pneumoniae infection can be of any serotype, such as a Streptococcus pneumoniae serotype 3, serotype 4, or serotype 19A infection. In some examples, the Streptococcus pneumoniae is serotype 3. The methods include administering to the subject an effective amount (that is, an amount effective to inhibit the infection in the subject) of a disclosed antibody, antigen binding fragment, multi-specific antibody, or a nucleic acid encoding such an antibody or antigen binding fragment, to a subject at risk of a Streptococcus pneumoniae infection or having the Streptococcus pneumoniae infection. The methods can be used pre-exposure or post-exposure. In some embodiments, the antibody or antigen binding fragment can be used in the form of a multi-specific antibody, such as bispecific antibody. The antigen binding fragment can be an scFv.

In some embodiments, a subject is selected for treatment. For example, a subject at risk of a Streptococcus pneumoniae infection or having the Streptococcus pneumoniae infection. The subject can have, or be at risk for pneumococcal disease, including, but not limited to, pneumococcal pneumonia, sepsis, or meningitis. Subjects with certain chronic conditions, such as diabetes, chronic obstructive pulmonary disease, cardiovascular diseases, and human immunodeficiency virus, are at increased risk of Streptococcus pneumoniae infection or pneumococcal disease. In some examples, the subject that is selected has diabetes, chronic obstructive pulmonary disease, cardiovascular diseases, and human immunodeficiency virus. The subject can have a co-infections with another virus, such as, but not limited to, an influenza virus.

In addition, acute conditions, such as a primary viral respiratory infection, also increase risk of Streptococcus pneumoniae infection. In some examples, the subject selected for treatment has an infection, other than a Streptococcus pneumoniae infection. For example, a subject can be selected that has a primary viral infection, such as a respiratory viral infection. In some embodiments, the subject that is selected has an influenza, coronavirus, respiratory syncytial virus, human metapneumovirus, parainfluenza virus infection. In specific, non-limiting examples, the infection is an influenza or severe acute respiratory syndrome coronavirus (SARS-CoV)-2 infection. In another non-limiting example, the infection is an influenza A infection.

Age is another risk factor, with subjects under the age of 2 years or over the age of 65 being more susceptible to Streptococcus pneumoniae infection. In some examples, the subject selected for treatment is under the age of about 2 years, or over the age of about 65 years. The subject can be less than one year of age, such as about 1, 2, 3, 4, 5, 6, 7, 8, 9, 10 or 11 months of age. The subject can be elderly, such as greater than about 65, 70, 75, 80, 85 or 90 years of age.

The infection does not need to be completely eliminated or inhibited for the method to be effective. For example, the method can decrease the infection by a desired amount, for example by at least 10%, at least 20%, at least 50%, at least 60%, at least 70%, at least 80%, at least 90%, at least 95%, at least 98%, or even at least 100% (elimination or prevention of detectable Streptococcus pneumoniae infection) as compared to the Streptococcus pneumoniae infection in the absence of the treatment. In some embodiments, the subject can also be treated with an effective amount of an additional agent, such as an anti-bacterial agent. In some embodiments, the subject is administered penicillin or a derivative thereof, such as amoxicillin, a macrolide, clindamycin, a cephalosporin, rifampin, vancomycin, trimethoprim-sulfamethoxazole, and/or ceftriaxone.

In some embodiments, administration of an effective amount of a disclosed antibody, antigen binding fragment, multi-specific antibody, or nucleic acid molecule encoding the disclosed antibody, inhibits the establishment of an infection and/or subsequent disease progression, such as the development of invasive pneumococcal disease, in a subject, which can encompass any statistically significant reduction in activity (for example, growth or invasion) or symptoms of the Streptococcus pneumoniae infection in the subject.

Methods are disclosed herein for the inhibition of Streptococcus pneumoniae replication in a subject. The methods include administering to the subject an effective amount (that is, an amount effective to inhibit replication in the subject) of a disclosed antibody, antigen binding fragment, multi-specific antibody (e.g., bispecific antibody), or a nucleic acid encoding such an antibody, antigen binding fragment, to a subject at risk of a Streptococcus pneumoniae infection or having a Streptococcus pneumoniae infection. The methods can be used pre-exposure or post-exposure.

Methods are disclosed for treating a Streptococcus pneumoniae infection in a subject. Methods are also disclosed for preventing a Streptococcus pneumoniae infection in a subject. These methods include administering one or more of the disclosed antibodies, antigen binding fragments, multi-specific antibodies, or nucleic acid molecules encoding such antibodies, or a composition including such antibodies, as disclosed herein. The nucleic acid molecules can be DNA or RNA.

Any route of administration is of use. In some embodiments, the antibody, multi-specific antibody (such as a bispecific antibody), antigen binding fragment, nucleic acid molecule, vector, or pharmaceutical composition is administered intranasally, intravenously, subcutaneously, intraperitoneally, or intramuscularly to the subject.

In some embodiments, the dose of the antibody, antigen binding fragment or multi-specific antibody can be from about 0.5 mg/kg to 50 mg/kg. In some examples, the dose is about 1 mg/kg to about 50 mg/kg, about 5 mg/kg to about 50 mg/kg, about 10 mg/kg to about 50 mg/kg, about 15 mg/kg to about 50 mg/kg, about 20 mg/kg to about 50 mg/kg, about 30 mg/kg to about 50 mg/kg, about 40 mg/kg to about 50 mg/kg, about 0.5 mg/kg to about 40 mg/kg, about 0.5 mg/kg to about 30 mg/kg, about 0.5 mg/kg to about 20 mg/kg, about 0.5 mg/kg to about 15 mg/kg, about 0.5 mg/kg to about 10 mg/kg, about 0.5 mg/kg to about 25 mg/kg, about 1 mg/kg to about 25 mg/kg, about 5 mg/kg to about 25 mg/kg, about 10 mg/kg to about 25 mg/kg, about 15 mg/kg to about 25 mg/kg, about 20 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 20 mg/kg, about 5 mg/kg to about 15 mg/kg, about 10 mg/kg to about 15 mg/kg, or about 10 mg/kg to about 20 mg/kg. In some examples, the dose is about 7.5 mg/kg to about 15 mg/kg.

Antibodies, antigen binding fragments thereof, and multi-specific antibodies can be administered by intravenous infusion. Doses of the antibody, antigen binding fragment, or multi-specific antibody vary, but generally range between about 0.5 mg/kg to about 50 mg/kg, such as a dose of about 1 mg/kg, about 5 mg/kg, about 7.5 mg/kg, about 10 mg/kg, about 15 mg/kg, about 20 mg/kg, about 30 mg/kg, about 40 mg/kg, or about 50 mg/kg. In some embodiments, the dose of the antibody, antigen binding fragment or multi-specific antibody can be from about 0.5 mg/kg to about 5 mg/kg, such as a dose of about 1 mg/kg, about 2 mg/kg, about 3 mg/kg, about 4 mg/kg or about 5 mg/kg. The antibody, antigen binding fragment, or multi-specific antibody is administered according to a dosing schedule determined by a medical practitioner. In some examples, the antibody, antigen binding fragment or multi-specific antibody is administered weekly, every two weeks, every three weeks or every four weeks.

Antibodies, antigen binding fragments thereof, and multi-specific antibodies (e.g., bispecific antibodies), can be administered intranasally. Doses of the antibody, antigen binding fragment, or multi-specific antibody vary, but generally range between about 0.5 mg/kg to about 50 mg/kg, such as a dose of about 1 mg/kg, about 5 mg/kg, about 7.5 mg/kg, about 10 mg/kg, about 15 mg/kg, about 20 mg/kg, about 30 mg/kg, about 40 mg/kg, or about 50 mg/kg. In some embodiments, the dose of the antibody, antigen binding fragment or multi-specific antibody can be from about 0.5 mg/kg to about 5 mg/kg, such as a dose of about 1 mg/kg, about 2 mg/kg, about 3 mg/kg, about 4 mg/kg or about 5 mg/kg.

The antibody, antigen binding fragment, or multi-specific antibody is administered according to a dosing schedule determined by a medical practitioner. In some examples, the antibody, antigen binding fragment or multi-specific antibody is administered weekly, every two weeks, every three weeks or every four weeks.

In some embodiments, the method of inhibiting the infection in a subject further comprises administration of one or more additional agents to the subject. Additional agents of interest include, but are not limited to, antibiotics, such penicillin or a derivative thereof, such as amoxicillin, a macrolide, clindamycin, a cephalosporin, rifampin, vancomycin, trimethoprim-sulfamethoxazole, and/or ceftriaxone.

In some embodiments, the method includes administering an effective amount of a combination of more than one of the disclosed antibodies or antigen binding fragments that specifically bind Streptococcus pneumoniae, or a nucleic acid molecule encoding such antibody or antigen binding fragment, for example, a combination of 2, 3, 4, 5, or more of the disclosed antibodies or antigen binding fragments, or nucleic acid molecules encoding the antibodies or antigen binding fragments. In some non-limiting examples, the method includes administering a first antibody or antigen binding fragment, for example, a PhtD3, PhtD6, PhtD7, PhtD8, or PspA16 antibody or antigen binding fragment, and a second antibody or antigen binding fragment, for example, another one of a PhtD3, PhtD6, PhtD7, PhtD8, or PspA16 antibody or antigen binding fragment. In further examples, the method includes administering a third antibody or antigen binding fragment, for example, another one of a PhtD3, PhtD6, PhtD7, PhtD8, or PspA16 antibody or antigen binding fragment.

In some embodiments, the first antibody or antigen binding fragment, binds one epitope of the PhtD or PspA, and an additional antibody or antigen binding fragment (e.g., a second, third, fourth, fifth antibody or antigen binding fragment), binds a different epitope of the PhtD or PspA. In some examples, each antibody in the combination binds a different epitope of PhtD or PspA. In some embodiments, one or more antibodies or antigen binding fragments that specifically bind PhtD, such as PhtD3, PhtD6, PhtD7, and/or PhtD8, is administered in combination with an antibody or antigen binding fragment that specifically binds PspA, such as PspA16. In a further example, at least one antibody or antigen binding fragment targeting PspA and at least two antibodies or antigen binding fragments targeting PhtD are administered, for example, PhtD3, PhtD7, and PspA16. Nucleic acid molecules encoding the above antibodies or antigen binding fragments, as disclosed herein, can also be administered to a subject in the disclosed methods.

In some embodiments, the method includes administering a PhtD3 antibody or antigen binding fragment. In further embodiments, the method includes administering a PhtD3 antibody or antigen binding fragment and one or more of: a PhtD7, PhtD6, PhtD8, and PspA16 antibody or antigen binding fragment. In one example, the method includes administering a PhtD3 antibody or antigen binding fragment and a PhtD7 antibody or antigen binding fragment. In another example, the method includes administering a PhtD3 antibody or antigen binding fragment and a PhtD6 antibody or antigen binding fragment. In a further example, the method includes administering a PhtD3 antibody or antigen binding fragment and a PhtD8 antibody or antigen binding fragment. In some examples, the method includes administering a PhtD3 antibody or antigen binding fragment and a PspA16 antibody or antigen binding fragment. Nucleic acid molecules encoding the antibodies or antigen binding fragments can also be administered to a subject in the disclosed methods.

In some embodiments, the method includes administering a PhtD7 antibody or antigen binding fragment. In further embodiments, the method includes administering a PhtD7 antibody or antigen binding fragment and one or more of: a PhtD3, PhtD6, PhtD8, and PspA16 antibody or antigen binding fragment. In one example, the method includes administering a PhtD7 antibody or antigen binding fragment and a PhtD6 antibody or antigen binding fragment. In another example, the method includes administering a PhtD7 antibody or antigen binding fragment and a PhtD8 antibody or antigen binding fragment. In a further example, the method includes administering a PhtD7 antibody or antigen binding fragment and a PspA16 antibody or antigen binding fragment. Nucleic acid molecules encoding the antibodies or antigen binding fragments can also be administered to a subject in the disclosed methods.

In some embodiments, the method includes administering a PhtD6 antibody or antigen binding fragment. In further embodiments, the method includes administering a PhtD6 antibody or antigen binding fragment and one or more of: a PhtD3, PhtD7, PhtD8, and PspA16 antibody or antigen binding fragment. In one example, the method includes administering a PhtD6 antibody or antigen binding fragment and a PhtD8 antibody or antigen binding fragment. In another example, the method includes administering a PhtD6 antibody or antigen binding fragment and a PspA16 antibody or antigen binding fragment. Nucleic acid molecules encoding the antibodies or antigen binding fragments can also be administered to a subject in the disclosed methods.

In some embodiments, the method includes administering a PhtD8 antibody or antigen binding fragment. In further embodiments, the method includes administering a PhtD8 antibody or antigen binding fragment and one or more of: a PhtD3, PhtD7, PhtD6, and PspA16 antibody or antigen binding fragment. In one example, the method includes administering a PhtD8 antibody or antigen binding fragment and a PspA16 antibody or antigen binding fragment. In some embodiments, the method includes administering a PspA16 antibody or antigen binding fragment. In further embodiments, the method includes administering a PspA16 antibody or antigen binding fragment and one or more of: a PhtD3, PhtD7, PhtD6, and PhtD8 antibody or antigen binding fragment. Nucleic acid molecules encoding the antibodies or antigen binding fragments can also be administered to a subject in the disclosed methods.

In some embodiments, a subject is administered DNA or RNA encoding a disclosed antibody, antigen binding fragment, or multi-specific antibody, to provide in vivo antibody production, for example using the cellular machinery of the subject. In some examples, an effective amount of mRNA encoding an scFV, or an mRNA encoding the V_{H} and V_{L} chain of antibody is administered to the subject. In some embodiments, the Fc domain of the antibody is modified to increase half-life, as disclosed above. Methods for administering exogenous mRNA for in vivo protein expression is disclosed, for example, in Schlake et al. (2019) Molecular Therapy 27(4): 773-784, which is incorporated by reference herein.

Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, antigen binding fragments thereof, or multi-specific antibody (e.g., bispecific antibody) can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol., 79(14), 8828-8834, 2005, which is incorporated by reference herein).

In several embodiments, a subject (such as a human subject at risk of a Streptococcus pneumoniae infection or having a Streptococcus pneumoniae infection) can be administered an effective amount of an AAV viral vector that comprises one or more nucleic acid molecules encoding a disclosed antibody, antigen binding fragment, or multi-specific antibody (e.g., bispecific antibody). The AAV viral vector is designed for expression of the nucleic acid molecules encoding a disclosed antibody, antigen binding fragment, or multi-specific antibody, and administration of the effective amount of the AAV viral vector to the subject leads to expression of an effective amount of the antibody, antigen binding fragment, or multi-specific antibody in the subject. Non-limiting examples of AAV viral vectors that can be used to express a disclosed antibody, antigen binding fragment, or multi-specific antibody in a subject include those provided in Johnson et al., Nat. Med., 15(8):901-906, 2009 and Gardner et al., Nature, 519(7541):87-91, 2015, each of which is incorporated by reference herein in its entirety.

In one embodiment, a nucleic acid encoding a disclosed antibody, antigen binding fragment, or multi-specific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody, antigen binding fragment, or multi-specific antibody (e.g., bispecific antibody), conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a Streptococcus pneumoniae infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The Streptococcus pneumoniae specific antibody, antigen binding fragment, or multi-specific antibody or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an embodiment, the antibody, antigen binding fragment, multi-specific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, multi-specific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of Streptococcus pneumoniae specific antibody, antigen binding fragment, conjugate, or nucleic acid molecule encoding such molecules, that are disclosed herein in a pharmaceutically acceptable carrier. In some embodiments, the composition comprises the PhtD3, PhtD6, PhtD7, PhtD8, or PspA16 antibody disclosed herein, or an antigen binding fragment thereof. In some embodiments, the composition comprises two, three, four or more antibodies or antigen binding fragments that specifically bind Streptococcus pneumoniae (e.g., PhtD3, PhtD6, PhtD7, PhtD8, or PspA16). In one example, the composition includes PhtD3 and PhtD7 antibody, or an antigen binding fragment. In other examples, the composition includes the antibody or antigen binding fragment of: PhtD3 and PhtD6, PhtD3 and PhtD8, PhtD3 and PspA16, PhtD7 and PhtD6, PhtD7 and PhtD8, PhtD7 and PspA16, PhtD6 and PhtD8, PhtD6 and PspA16, or PhtD8 and PspA16. In some embodiments, at least one antibody, antigen binding fragment, or multi-specific antibody that specifically binds PhtD is included in the composition (e.g., PhtD3, PhtD6, PhtD7, or PhtD8). In other embodiments, at least one antibody, antigen binding fragment or multi-specific antibody that specifically binds PspA in included in the composition (e.g., PspA16). The compositions are useful, for example, for the inhibition or detection of a Streptococcus pneumoniae infection, such as a Streptococcus pneumoniae serotype 3, serotype 4, or serotype 19A infection.

The compositions can be prepared in unit dosage forms, such as in a kit, for administration to a subject. The amount and timing of administration are at the discretion of the administering physician to achieve the desired purposes. The antibody, antigen binding fragment, multi-specific (e.g. bispecific) antibody, conjugate, or nucleic acid molecule encoding such molecules can be formulated for systemic or local administration. In one example, the, antigen binding fragment, multi-specific antibody, conjugate, or nucleic acid molecule encoding such molecules, is formulated for parenteral administration, such as intravenous administration, or intranasal administration.

In some embodiments, the antibody, antigen binding fragment, multi-specific antibody, or conjugate thereof, in the composition is at least 70% (such as at least 75%, at least 80%, at least 85%, at least 90%, at least 95%, at least 96%, at least 97%, at least 98% or at least 99%) pure. In some embodiments, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, multi-specific antibody (e.g., bispecific antibody), conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody, antigen binding fragment, multi-specific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation including one or more antibodies, antigen binding fragments, or multi-specific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

A typical composition for intranasal administration comprises about 0.01 to about 50 mg/kg of antibody, antigen binding fragment, multi-specific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation or nebulized including one or more antibodies, antigen binding fragments, or multi-specific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml. In some embodiments, a nebulizer is used.

An aerosol composition is provided for the delivery of the antibody, an antigen binding fragment thereof, or a multi-specific antibody (e.g., bispecific antibody). Exemplary methods for nebulization of monoclonal antibodies are disclosed in European Publication No. EP18819934. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung.

Pulmonary delivery can be achieved by inhalation, and administration by inhalation herein may be oral and/or nasal. Examples of pharmaceutical devices for pulmonary delivery include metered dose inhalers (MDIs), dry powder inhalers (DPIs), and nebulizers. Exemplary delivery systems by inhalation which can be adapted for delivery of a therapeutic agent (such as an antibody, an antigen binding fragment thereof, a multi-specific antibody (e.g., bispecific antibody)) are described in, for example, U.S. Pat. Nos. 5,756,353; 5,858,784; and PCT Publication Nos. WO98/31346; WO98/10796; WO00/27359; WO01/54664; WO02/060412. Aerosola formulations of use for delivering antibodies are also disclosed in U.S. Pat. Nos. 6,294,153; 6,344,194; 6,071,497, and PCT Publication No. WO02/066078; PCT Publication No. WO02/053190; PCT Publication No. WOO 1/60420; and PCT Publication No. WO00/66206.

Pressurized metered dose inhalers (pMDIs) can be used. In these devices, an aerosol is created when a valve is opened (usually by pressing down on the propellant canister), allowing liquid propellant to spray out of a canister. Typically, a therapeutic agent is contained in small particles (usually a few microns in diameter) suspended in the liquid propellant, but in some formulations the therapeutic agent can be dissolved in the propellant. The propellant evaporates rapidly as the aerosol leaves the device, resulting in small particles that are inhaled. Propellants typically used in such pMDIs include but are not limited to hydrofluoroalkanes (HFAs). A surfactant can also be used, for example, to formulate the therapeutic agent, with pMDIs. Other solvents or excipients can also be employed with pMDIs, such as ethanol, ascorbic acid, sodium metabisulfate, glycerin, chlorobutanol, and cetylpyridium chloride. Such pMDIs can further include add-on devices such as, for example, spacers, holding chambers and other modifications.

Nebulizers produce a mist of drug-containing liquid droplets for inhalation and can be classified into two types: ultrasonic nebulizers and jet nebulizers. In addition, single breath atomizers have also been developed (e.g., RESPIMAT®), which deliver a therapeutic agent, such as an antibody, an antigen binding fragment, or a multi-specific antibody (for example, a bi-specific antibody) in a single inhalation. Jet nebulizers use a source of pressurized air to blast a stream of air through a therapeutic agent-containing water reservoir, producing droplets in a complex process involving a viscosity-induced surface instability that leads to nonlinear phenomena in which surface tension and droplet breakup on baffles play a role. Ultrasonic nebulizers produce droplets by mechanical vibration of a plate or mesh. In either type of nebulizer, the therapeutic agent can be contained in solution in the liquid in the nebulizer and so the droplets being produced contain the therapeutic agent in solution. However, the therapeutic agent also can be contained in small particles suspended in the water, which are then contained as particles suspended inside the droplets being produced. Certain excipients are usually included in formulations suitable for nebulization, such as sodium chloride (e.g., to maintain isotonicity), mineral acids and bases (e.g., to maintain or adjust pH), nitrogen headspace sparging, benzalkonium chloride, calcium chloride, sodium citrate, disodium edtate, and polysorbate 80.

Another type of inhaler is a dry powder inhaler (DPI). In DPIs, the aerosol is can be a powder, contained within the device until it is inhaled. The therapeutic agent is manufactured in powder form as small powder particles (usually a few millionths of a meter, or micrometers, in diameter). In many DPIs, the therapeutic agent is mixed with much larger sugar particles (e.g., lactose monohydrate), that are typically 50-100 micrometers in diameter. The increased aerodynamic forces on the lactose/therapeutic agent agglomerates can improve entrainment of the particles upon inhalation. Upon inhalation, the powder is broken up into its constituent particles with the aid of turbulence and/or mechanical devices such as screens or spinning surfaces on which particle agglomerates impact, releasing the small, individual therapeutic agent/powder particles into the air to be inhaled into the lung. The sugar particles can be left behind in the device and/or in the mouth-throat.

Antibodies, an antigen binding fragment thereof, a multi-specific antibody (e.g., bispecific antibody), or a nucleic acid encoding such molecules, can be provided in lyophilized form and rehydrated with sterile water before administration, although they are also provided in sterile solutions of known concentration. A solution including the antibody, antigen binding fragment, multi-specific antibody, or a nucleic acid encoding such molecules, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 m are generally referred to as nanoparticles, nanospheres, and nanocapsules, respectively. Capillaries have a diameter of approximately 5 m so that only nanoparticles are administered intravenously. Microparticles are typically around 100 m in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 2. Methods of Detection and Diagnosis

Methods are also provided for the detection of the presence of Streptococcus pneumoniae in vitro or in vivo. In one example, the presence of Streptococcus pneumoniae is detected in a biological sample from a subject and can be used to identify a subject with an infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody, antigen binding fragment, or multi-specific antibody (e.g., bispecific antibody), that specifically binds to Streptococcus pneumoniae, or conjugate thereof (e.g., a conjugate including a detectable marker) under conditions sufficient to form an immune complex, and detecting the immune complex (e.g., by detecting a detectable marker conjugated to the antibody or antigen binding fragment).

In one embodiment, the antibody, antigen binding fragment or multi-specific antibody is directly labeled with a detectable marker. In another embodiment, the antibody (or antigen binding fragment or multi-specific antibody) that binds Streptococcus pneumoniae (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment, multi-specific antibody or secondary antibody are known and described above, and include various enzymes, prosthetic groups, fluorescent materials, luminescent materials, magnetic agents and radioactive materials.

In some embodiments, the disclosed antibodies, antigen binding fragments thereof, or multi-specific antibodies are used to test vaccines. For example, to test if a vaccine composition including PhtD or PspA or fragment thereof assumes a conformation including the epitope of a disclosed antibody. Thus, provided herein is a method for testing a vaccine, wherein the method comprises contacting a sample containing the vaccine, such as a PhtD or PspA immunogen, with a disclosed antibody, antigen binding fragment, or multi-specific antibody, under conditions sufficient for formation of an immune complex, and detecting the immune complex, to detect the vaccine including the epitope of interest in the sample. In one example, the detection of the immune complex in the sample indicates that vaccine component, such as the immunogen assumes a conformation capable of binding the antibody or antigen binding fragment.

### EXAMPLES

Human mAbs were isolated (PhtD3, PhtD6, PhtD7, PhtD8, and PspA16) that specifically bind pneumococcal histidine triad protein (PhtD) or pneumococcal surface protein A (PspA), two conserved and protective antigens. mAbs to PhtD target diverse epitopes spanning the entire PhtD protein, and mAb PspA16 targets the N-terminal segment of PspA. The PhtD-specific mAbs were found to bind to multiple serotypes, while PspA16 serotype breadth was limited. The prophylactic efficacy of two PhtD mAbs was examined for pneumococcal disease prevention, PhtD3 and PhtD8, which target the N-terminal and C-terminal regions of PhtD, respectively. While both mAbs prolonged the survival of mice infected with pneumococcal serotype 3, PhtD3 provided more robust protection. To examine the breadth of protection induced by mAb PhtD3, the mAb were tested in a prophylactic treatment study against serotype 4, and PhtD3 prolonged survival of mice in both intranasal and intravenous infection models. Furthermore, mAb PhtD3 was protective in a serotype 3 treatment model when administered 24 hours after pneumococcal infection. All PhtD and PspA mAbs demonstrated opsonophagocytic activity. These results provide new human mAbs for disease prevention and treatment, and identify epitopes on PhtD and PspA recognized by human B cells for therapeutic and vaccine development.

### Example 1

### Materials and Methods

### Blood Draws and PBMC Isolation

90 mL of blood was drawn by venipuncture into 9 heparin-coated tubes, and 10 mL of blood was collected into a serum separator tube. Peripheral blood mononuclear cells (PBMCs) were isolated from human donor blood samples using Ficoll-Histopaque density gradient centrifugation, and PBMCs were frozen in the liquid nitrogen vapor phase until further use.

### Pneumococcal Protein Cloning and Expression

PspA and PhtD full-length proteins and fragments were cloned from the genome of S. pneumoniae strain TCH8431 (serotype 19A) with primers listed in Table 2 below. The full-length PspA and PhtD were ligated into the pET28a vector while the fragments were ligated into the pMAL-c5× vector. The sequences of all constructed plasmids were confirmed by sequencing, and then transformed into E. coli BL21(DE3) for protein expression. Single colonies of transformed E. coli were picked and cultured in 5 mL of LB medium supplemented with antibiotic (50 μg/ml kanamycin for pET28a, 100 μg/ml ampicillin for pMAL-c5x) overnight in a shaking incubator at 37° C. The overnight culture was then expanded at a 1:100 ratio in 2×YT medium with antibiotic and cultured at 37° C. After the OD₆₀₀ reached 0.5 to 0.7, the culture was induced with 50 μM isopropyl-D-thiogalactopyranoside for 12-16 hrs at room temperature. Bacteria pellets collected by centrifugation at 6,000×g for 10 min, and frozen at −80° C. Thawed E. coli pellets were resuspended in 10 mL of buffer containing 20 mM Tris pH 7.5 and 500 mM NaCl, and then lysed by sonication. Cell lysates were centrifuged at 12,000×g for 30 min and the supernatant was subsequently used for protein purification through HisTrap™ column (His-tagged full-length proteins, GE Healthcare® or Amylose resin (MBP-tagged fragments, New England Biolabs) by following manufacturer's protocols.

TABLE 2
Summary of primers used for cloning
of PspA and PhtD genes.

| Primer | SEQ ID NO | Sequences (5′ −> 3′) |
|---|---|---|
| PspA1-F | SEQ ID NO: 51 | CGCCATATGatggctaataagaaaaa |
| | | aatgatttt |
| PspA247-F | SEQ ID NO: 52 | CGCCATATGgagctaaacgctaaaca |
| | | a |
| PspA436-F | SEQ ID NO: 53 | CGCCATATGgatgaagaagaaactcc |
| | | agcg |
| PspA438-R | SEQ ID NO: 54 | TAGCGGCCGTTAATGGTGATGGTGAT |
| | | GGTGttcttcatctccatcagggc |
| PspA512-R | SEQ ID NO: 55 | TAGCGGCCGTTAATGGTGATGGTGAT |
| | | GGTGttttggagtggctggtttttc |
| PspA725-R | SEQ ID NO: 56 | TAGCGGCCGTTAATGGTGATGGTGAT |
| | | GGTGaacccattcaccattggcat |
| PhtD1-F | SEQ ID NO: 57 | CATGCCATGGCCatgaaaatcaataa |
| | | aaaatatctagcagg |
| PhtD168-F | SEQ ID NO: 58 | CATGCCATGGCCgcagataatgctgt |
| | | tgctg |
| PhtD341-F | SEQ ID NO: 59 | CATGCCATGGCCtatcgttcaaacca |
| | | ttgggt |
| PhtD645-F | SEQ ID NO: 60 | CATGCCATGGCCgaccattaccataa |
| | | catcaaatttg |
| PhtD170-R | SEQ ID NO: 61 | CCCAAGCTTTTAATGGTGATGGTGAT |
| | | GGTGattatctgctcttgagttatga |
| | | ttatg |
| PhtD343-R | SEQ ID NO: 62 | CCCAAGCTTTTAATGGTGATGGTGAT |
| | | GGTGtgaacgataacgaaggggaat |
| PhtD647-R | SEQ ID NO: 63 | CCCAAGCTTTTAATGGTGATGGTGAT |
| | | GGTGgtaatggtcataatgaggtatg |
| | | attaaa |
| PhtD838-R | SEQ ID NO: 64 | CCCAAGCTTTTAATGGTGATGGTGAT |
| | | GGTGctgtataggagccggttga |

*Restriction enzyme sites are in bold, His-tags are underlined.

### Enzyme-Linked Immunosorbent Assay for Binding to Pneumococcal Proteins

For recombinant protein capture ELISA assays, 384-well plates were treated with 2 μg/ml of antigen in PBS for 1 hr at 37° C. or overnight at 4° C. Following this, plates were washed once with water before blocking for 1 hr with 2% nonfat milk/2% goat serum in PBS-T (blocking buffer). Plates were washed with water three times before applying serially diluted primary mAbs in PBS for 1 hr. Following this, plates were washed with water three times before applying 25 μl of secondary antibody (goat anti-human IgG Fc; Meridian Life Science®) at a dilution of 1:4,000 in blocking solution. After incubation for 1 hr, the plates were washed five times with PBS-T, and 25 μl of a PNPP (p-nitrophenyl phosphate) solution (1 mg/ml PNPP in 1 M Tris base) was added to each well. The plates were incubated at room temperature for 1 hr before reading the optical density at 405 nm on a BioTek® plate reader. Binding assay data were analyzed in GraphPad Prism using a nonlinear regression curve fit and the log(agonist)-versus-response function to calculate the binding EC50 values.

### Generation of Pneumococcal-Specific Hybridomas

For hybridoma generation, 10 million peripheral blood mononuclear cells purified from the blood of human donors were mixed with 8 million previously frozen and gamma irradiated NIH 3T3 cells modified to express human CD40L, human interleukin-21 (IL-21), and human BAFF(69) in 80 mL StemCell™ medium A (StemCell Technologies™) containing 6.3 μg/mL of CpG (phosphorothioate-modified oligodeoxynucleotide from Invitrogen®; see PCT Publication No. WO 2017/011394A1, and Bar-Peled et al., J Virol (2019) doi:10.1128/jvi.00342-19, both incorporated herein by reference) and 1 μg/mL of cyclosporine A (Millipore-Sigma®). The mixture of cells was plated in four 96-well plates at 200 μl per well in StemCell™ medium A. After 6 days, culture supernatants were screened by ELISA for binding to recombinant pneumococcal protein, and cells from positive wells were electrofused to generate hybridomas and biologically cloned as previously described (Bar-Peled et al. (2019) J Virol 93:e00342-19).

### Human mAb Expression and Purification

For hybridoma-derived mAbs, hybridoma cell lines were expanded in StemCell™ medium A until 80% confluent in 75-cm² flasks. Cells from one 75-cm² cell culture flask were collected with a cell scraper and expanded to 225-cm² cell culture flasks in serum-free medium (Hybridoma-SFM; Thermo Fisher Scientific®). Recombinant cultures from transfection were stopped after 5 to 7 days, hybridoma cultures were stopped after 30 days. For recombinant PhtD3-IgG₂ₐ, plasmids encoding cDNAs for the heavy and light chain sequences of PhtD3-IgG₂ₐ were synthesized (GenScript®), and cloned into pCDNA3.1+. mAbs were obtained by transfection of plasmids into Expi293F™ cells by transfection. For each milliliter of transfection, 1 μg of plasmid DNA was mixed with 4 μg of 25,000-molecular-weight polyethylenimine (PEI; PolySciences, Inc.®) in 66.67 μl Opti-MEM® cell culture medium (Gibco®). After 30 min, the DNA-PEI mixture was added to the Expi293F™ cells, and cells were cultured for 5-6 days for protein expression. Culture supernatants from both approaches were filtered using 0.45 μm filters to remove cell debris. mAbs were purified directly from culture supernatants using HiTrap protein G columns (GE Healthcare Life Sciences®) according to the manufacturer's protocol.

Isotype Determination for Human mAbs

For determination of mAb isotypes, 96-well Immulon® 4HBX plates (Thermo Fisher Scientific®) were coated with 2 μg/mL of each mAb in PBS (duplicate wells for each sample). The plates were incubated at 4° C. overnight and then washed once with water. Plates were blocked with blocking buffer and then incubated for 1 hr at room temperature. After incubation, the plates were washed three times with water. Isotype-specific antibodies obtained from Southern Biotech® (goat anti-human kappa-alkaline phosphatase [AP] [catalog number 100244-340], goat anti-human lambda-AP [catalog number 100244-376], mouse anti-human IgG1 [Fc]-AP [catalog number 100245714], mouse anti-human IgG2 [Fc]-AP [catalog number 100245-734], mouse anti-human IgG3 [hinge]-AP [catalog number 100245-824], and mouse anti-human IgG4 [Fc]-AP [catalog number 100245-812]) were diluted 1:1,000 in blocking buffer, and 50 μl of each solution was added to the respective wells. Plates were incubated for 1 h at room temperature and then washed five times with PBS-T. The PNPP substrate was prepared at 1 mg/mL in substrate buffer (1 M Tris base, 0.5 mM MgCl₂, pH 9.8), and 100 μl of this solution was added to each well. Plates were incubated for 1 hr at room temperature and read at 405 nm on a BioTek® plate reader.

### RT-PCR for Hybridoma mAb Variable Gamma Chain and Variable Light Chain.

RNA was isolated from expanded hybridoma cells using the E.Z.N.A.® total RNA kit (Omega BioTek®) according to the manufacturer's protocol. cDNA was obtained using the SuperScript™ IV Reverse Transcriptase kit. Following this, PCR was conducted in two steps using established primers for the heavy chain, and kappa and lambda light chains (Guthmiller et al. (2019) An Efficient Method to Generate Monoclonal Antibodies from Human B Cells BT—Human Monoclonal Antibodies: Methods and Protocols, p. 109-145. In Steinitz, M (ed.) Springer New York, New York, NY). Samples were analyzed by agarose gel electrophoresis and purified PCR products (E.Z.N.A.® Cycle-Pure kit; Omega Biotek®) were cloned into the pCR2.1 vector using the Original TA Cloning® kit (Thermo Fisher Scientific®) according to the manufacturer's protocol. Plasmids were purified from positive DH5α colonies with E.Z.N.A.® plasmid DNA mini kit (Omega Biotek®) and submitted to Genewiz® for sequencing. Sequences were analyzed using IMGT/V-Quest (Brochet et al. (2008) Nucleic Acids Res 36:503-508).

### Experimental Setup for Biolayer Interferometry

For all biosensors, an initial baseline in running buffer (PBS, 0.5% bovine serum albumin [BSA], 0.05% Tween 20, 0.04% thimerosal) was obtained. For epitope mapping, 100 μg/mL of His-tagged PhtD protein was immobilized on anti-penta-HIS biosensor tips (FortdBio®) for 120 s. For binding competition, the baseline signal was measured again for 60 s before biosensor tips were immersed into wells containing 100 μg/mL of primary antibody for 300 s. Following this, biosensors were immersed into wells containing 100 μg/mL of a second mAb for 300 s. Percent binding of the second mAb in the presence of the first mAb was determined by comparing the maximal signal of the second mAb after the first mAb was added to the maximum signal of the second mAb alone. mAbs were considered noncompeting if maximum binding of the second mAb was >66% of its uncompeted binding. A level of between 33% and 66% of its uncompeted binding was considered intermediate competition, and <33% was considered competition.

### Bacterial Strains and Growth Conditions

Pneumococcal strains were grown at 37° C. in 5% CO₂ in Todd-Hewitt broth (BD®, Franklin Lakes NJ) supplemented with 0.5% yeast extract for 12 hrs. Ten percent glycerol was added to the media and 500 μL aliquots were made. Cultures were kept at −80° C. until used, cultures were washed twice with PBS before being used in experiments. Colonies were grown on BD® Trypticase™ Soy Agar II with 5% Sheep Blood (BD®, Franklin Lakes NJ). The numbers of CFUs per milliliter of these stocks were determined, after the aliquots had been frozen, by plating a single quick-thawed diluted aliquot on sheep's blood agar plates. The calculated number of CFUs was subsequently used to make dilutions for experiments from aliquots thawed at later times. In each experiment, the actual number of CFUs administered was determined by plating on blood agar at the time of the assay. Strains used in this study are listed in Table 3.

TABLE 3
Summary of pneumococcal strains used in this study.

| Pneumococcal Strain | Serotype | Source |
|---|---|---|
| SPEC 1 | 1 | BEI NR-13388 |
| STREP2 | 2 | BEI NR-31700 |
| WU2 | 3 | Gift from Moon Nahm, UAB |
| TIGR4 | 4 | Gift from Larry McDaniel, UMiss |
| SPEC6C | 6C | BEI NR-20805 |
| SPEC6D | 6D | BEI NR-20806 |
| STREP8 | 8 | BEI NR-31701 |
| SPEC9N | 9N | BEI NR-31702 |
| OREP10A | 10A | BEI NR-31703 |
| TREP11A | 11A | BEI NR-31705 |
| TREP12F | 12F | BEI NR-31704 |
| TREP15B | 15B | BEI NR-33666 |
| OREP17F | 17F | BEI NR-31706 |
| TCH8431 | 19A | BEI HM-145 |
| SPEC20B | 20B | BEI NR-33664 |
| TREP22F | 22F | BEI NR-31707 |
| STREP33F | 33F | BEI NR-33665 |

### Western Blot

Pneumococcal strains were mixed with non-reducing loading buffer (Laemmli SDS sample buffer, non-reducing 6X) and loaded on a 4-12% Bis-Tris gel (Invitrogen®). Samples were then transferred to PVDF membranes via iBlot® system (Invitrogen®) and then blocked with 5% blocking buffer (5% nonfat milk in PBS-T) for 1 hr at room temperature or at 4° C. overnight. The membrane was washed three times in five-minute intervals on an orbital shaker with 0.05% PBS-T. Then, primary antibodies were added at dilutions of 1 μg/mL in PBS for one hour at room temperature. The membranes were then washed three time in five-minute intervals with PBS-T on an orbital shaker, and soaked in the secondary antibody at a 1:8,000 dilution in blocking buffer for one hour. Next, the membranes were then washed five times in five-minute intervals on the orbital shaker with PBS-T, and substrate (Pierce™ ECL Western Blotting Substrate, Thermo Scientific®) was added and an image was taken immediately with the ChemiDoc™ Imaging System (BioRad®).

### Enzyme-Linked Immunosorbent Assay of Fixed Pneumococcus.

384-well plates were treated with 15 μL (˜10⁷ CFUs) of whole cell pneumococcus in PBS into each well. Cell density was checked by microscope to ensure a confluent layer of pneumococcus was coated. The bacteria were then fixed with 15 μl of 4% paraformaldehyde into each well and placed onto a plate shaker for 10 mins to mix. The 384-well plates were incubated at 4° C. for 24-48 hours to allow the bacteria to fix to the bottom of the plates. Following this, the plates were washed once with 75 μl of PBS-T into each well. The plates were then blocked with 2% blocking buffer for 1 hr at room temperature then washed three times with PBS-T. Next, 25 μl of serially diluted primary antibodies were applied to the wells for 1 hr at room temperature, then plates were washed with PBS-T three times. Following this, 25 μl of secondary antibody (goat anti-human IgG Fc; Meridian Life Science®) at a 1:4,000 dilution in blocking buffer was applied to each well for 1 hr at room temperature. After the plates were washed with PBS-T five times, 25 μl of PNPP (p-nitrophenyl phosphate) solution (1 mg/ml PNPP in 1 M Tris bases) was added to each well for 1 hr at room temperature. After 1 hr the optical density was read at 405 nm on a BioTek® plate reader. Binding assay data were analyzed in GraphPad Prism.

### Binding of Antibodies to Bacteria by Flow Cytometry

The ability of mAbs to bind antigen exposed on the surface of S. pneumoniae was determined by flow cytometry. Bacteria were stained with 10 μM CFSE (Millipore-Sigma®) for 1 hr at 37° C. Bacteria were then washed with Hank's Balanced Salt Solution (HBSS) containing 1% bovine serum albumin (BSA) to remove excess stain. Following this, 1×10⁶ bacteria were incubated with 10 μg/ml of antibody for 30 min at 37° C. Bacteria were then washed twice with HBSS+1% BSA. Antibody binding was detected using an APC Anti-Human IgG Fc (Biolegend™) at a 1:100 dilution incubated for 1 hr with the bacteria. Cells were washed with HBSS+1% BSA and fixed in 2% paraformaldehyde (PFA) in PBS prior to analysis on a NovoCyte Quanteon™ Flow Cytometer.

### Determination of PhtD3 Efficacy

For lethal pneumonia challenge studies, 5-7-week-old CBA/CaHN-Btkxid/J (CBA/N) mice (The Jackson Laboratory®, Bar Harbor, ME) were used. Mice were intraperitoneally inoculated with antibody treatments two hours prior to pneumococcal infection. For infection, mice were anesthetized by inhalation of 5% isoflurane and intranasally challenged with 30 μL of PBS containing 10⁵ colony-forming units (CFUs) of TIGR4. Actual doses delivered to mice were determined by titering the bacteria after delivery. Mice were weighed and assessed daily, and were considered moribund when >20% of body weight was lost or they were not responsive to manual stimulation. Mice were euthanized by CO₂ asphyxiation followed by cervical dislocation. For the sepsis model, C57BL/6 mice 5-7 weeks old (Charles River Laboratories®) were used. Mice were intraperitoneally inoculated with antibody treatments two hours prior to pneumococcal infection. Mice infected intravenously with 10⁶ CFUs of TIGR4 via the tail vein. Mice were monitored and euthanized as described above.

### Opsonophagocytic Killing Assay

An opsonophagocytic killing assay was performed as described previously as adapted from an earlier protocol with modifications (Burton and Nahm (2012) Clin Vaccine Immunol 2012/04/18. 19:835-841). TIGR4 stocks were incubated in triplicate wells in a 96-well round-bottom plate for 1 hour at 37° C. with the indicated antibodies (10 μg of antibody per well in a final volume of 100 μL per well) in opsonization buffer B (OBB: sterile 1×PBS with Ca²⁺/Mg²⁺, 0.1% gelatin, and 5% heat-inactivated FetalClone™ HyClone™, with heat-inactivated FetalClone™-treated only TGR4 cells serving as a control. Cells of the human promyelocytic leukemia cell line HL-60 (ATCC) were cultured in RPMI with 10% heat-inactivated FetalClone™ and 1% 1-glutamine. HL-60 cells were differentiated using 0.6% N,N-dimethylformamide (DMF [Fisher®]) for 3 days before performing the OPA assay, harvested, and resuspended in OBB. Baby rabbit complement (Pel-Freez®) was added to HL-60 cells at a 1:5 final volume. The HL-60-complement mixture was added to the bacteria at 5×10⁵ cells/well. The final reaction mixtures were incubated at 37° C. for 1 hour with shaking. The reactions were stopped by incubating the samples on ice for approximately 20 min. Then 10 μL of each reaction mixture (triplicate) was diluted to a final volume of 50 μL and plated onto blood agar plates. Plates were incubated overnight at 30° C. and counted the next day. The percentage of bacterial killing was calculated as each sample replicate normalized to the mean value obtained for the control samples, subtracted from 100 (with No Ab control samples representing 0% survival).

### Flow-Based Opsonophagocytosis Assay

Pneumococcal cells were stained with PHrodo™ Succinimidyl Ester (Invitrogen®) following manufacturer's protocol. Approximately, ˜10⁸ CFUs of bacteria were fixed with 1% paraformaldehyde in PBS for 30 min at room temperature. Fixed bacteria were washed twice with PBS and resuspended with 0.5 mL freshly prepared 100 mM NaHCO₃ (pH 8.5). Immediately before use, the contents of a 0.1 mg vial of pHrodo™ iFL amine-reactive dye were dissolved in 10 μL of DMSO to prepare a 10 mM stock solution. pHrodo™ was diluted in the bacterial suspension at a final concentration of 0.1 mM, and bacteria were stained for 1 hr at room temperature. Stained bacteria were washed twice with Hank's Balanced Salt Solution with Ca²⁺ and Mg²⁺ (HBSS, Gibco®), and resuspended with 0.5 mL HBSS and stored in the dark at 4° C. The opsonophagocytosis assay was performed in 96 well U-bottom plates in a total volume of 120 μL per well. First, 20 μL of pHrodo™ labeled bacteria (˜10⁷ CFUs/well) was mixed with 40 μL of sterile filtered mAbs (50 pig/well), and incubated on a shaker at 37° C. for 30 min. Bacteria were mixed with HBSS as a negative control, and purified human serum IgG was used as a positive control. Differentiated HL-60 cells were washed twice with HBSS and mixed with baby rabbit complement (Pel-Freez Biologicals®) at a final concentration of 10% in each well. Following this, 60 μL (1×10⁶ viable cells) of differentiated HL60 cells and complement were added to the mixture of bacteria and antibodies, and incubated on a shaker at 37° C. for 60 min. The plate was then centrifuged at 1300 rpm for 5 min at 4° C. to remove the supernatant and the pellet was washed twice with 200 μL of HBSS. After the second wash, the pellet was resuspended in a 50 μL mixture of PE-anti-human CD11b (Southern Biotech®, 10 μL/million cells), Alexa Fluor 647®-anti-human CD35 (BD Biosciences®, 5 μL/million cells), and DAPI (Invitrogen®, 50 ng/million cells) in PBS containing 1% BSA. After a 30 min incubation at 4° C. in the dark, the plate was washed twice with 200 μl of PBS, and cells were resuspended in 100 μL of PBS. Cells were analyzed with a NovoCyte Quanteon™ Flow Cytometer. Single fluorophore stained differentiated HL60 cells and pHrodo™ stained bacteria were used to calculate the compensation matrix. A total of 10,000 ungated events were collected from each sample well, and data were analyzed by FlowJo®.

### Example 2

### Isolation and Characterization of Pneumococcal Protein-Specific Human mAbs

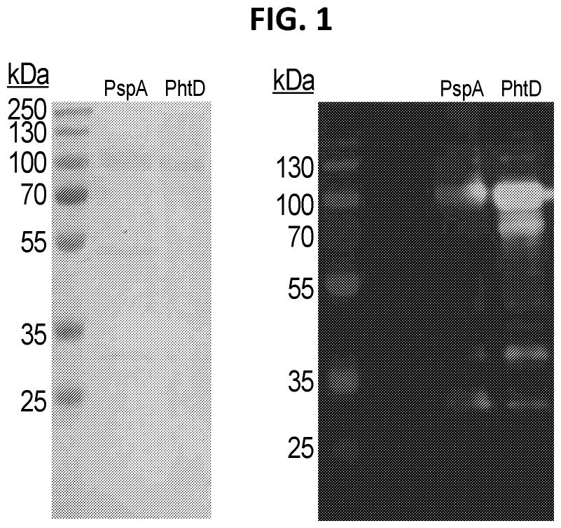

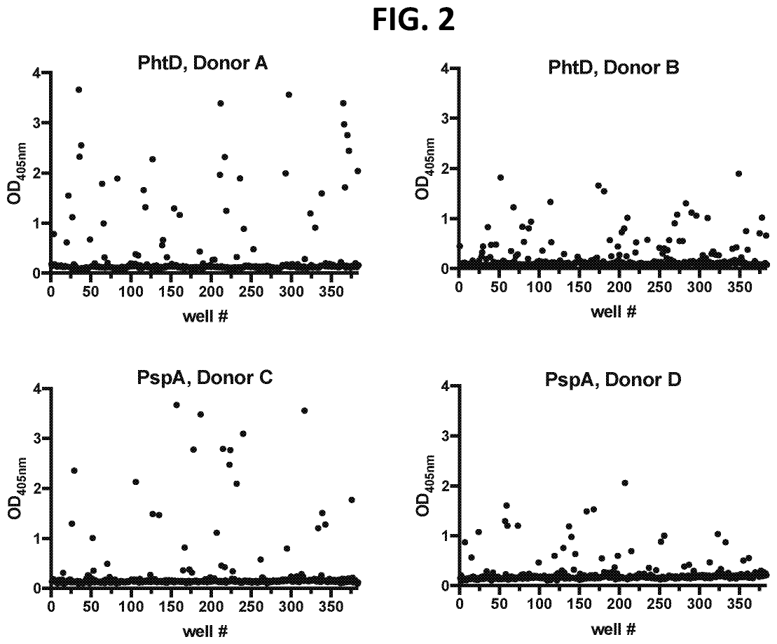

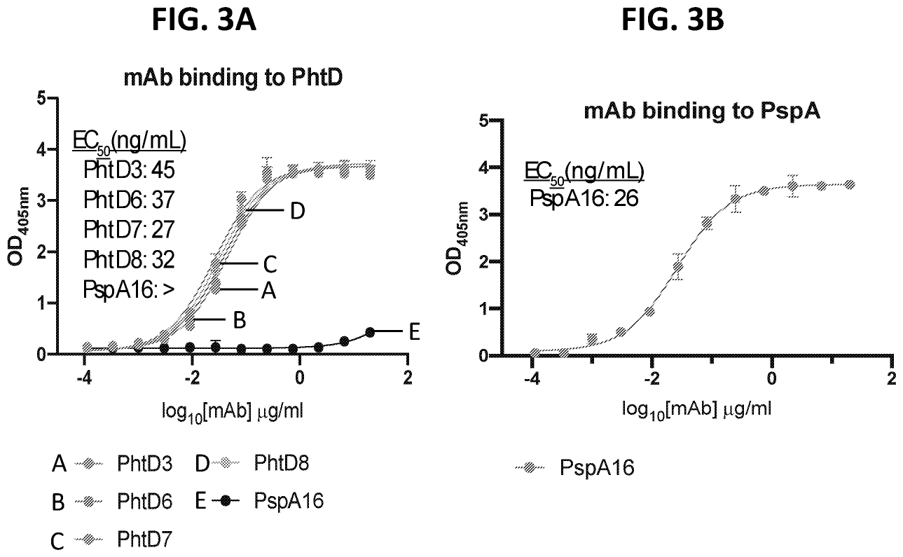

To identify PhtD and PspA-specific human mAbs, His-tagged PhtD and PspA from strain TCH8431 (serotype 19A) was recombinantly expressed in E. coli (FIG. 1), and these proteins were utilized to screen stimulated B cells from human donor peripheral blood mononuclear cells (PBMCs) as previously described (Bar-Peled et al. (2019) J Virol 93:e00342-19). PBMCs from healthy human subjects were plated onto a feeder layer expressing human CD40L, human IL-21, and human BAFF for six days to stimulate B cell growth and antibody secretion. Cell supernatants from the stimulated B cells were screened against recombinant PhtD and PspA by enzyme-linked immunosorbent assay. Responses to the recombinant proteins were varied between subjects as shown in an example in FIG. 2. From five subject PBMCs, several reactive wells were fused for generation of human hybridomas and subsequent human mAb isolation. Four hybridomas lines, each from unique donors, were successfully generated and biologically cloned by single cell sorting for PhtD, and one mAb was generated for PspA from an independent subject. Each of the mAbs to PhtD had similar binding EC₅₀ values determined by ELISA (FIG. 3A), and mAbs to PhtD and PspA bound with high avidity with EC₅₀ values ranging from 26-45 ng/mL (FIGS. 3A and 3B). To determine the V, D, and J genes utilized by each mAb, the hybridomas were sequenced by RT-PCR followed by TA cloning and the results are shown in Table 4.

TABLE 4
Summary of genetic characteristics of pneumococcal-specific mAbs.

| | | V_{H} gene | | | HCDR3 | V_{L} gene | | LCDR3 |
|---|---|---|---|---|---|---|---|---|
| mAb | isotype | (% mutation) | D_{H} | J_{H} | sequence | (% mutation) | J_{L} | sequence |
| PhtD3 | IgG₁, k | V1-69*18 | D3- | J3*01 | SEQ ID | V3-20*01 | J4*01 | SEQ ID |
| | | (85%) | 16*01 | | NO: 4 | (91%) | | NO: 8 |
| PhtD6 | IgG₁, k | V1-8*01 | D2- | J5*02 | SEQ ID | V1-39*01 | J1*01 | SEQ ID |
| | | (93%) | 15*01 | | NO: 20 | (92%) | | NO: 24 |
| PhtD7 | IgG1, λ | V1-2*02 | D3- | J4*02 | SEQ ID | V4-69*01 | J3*02 | SEQ ID |
| | | (91%) | 16*01 | | NO: 28 | (88%) | | NO: 32 |
| PhtD8 | IgG₁, λ | V1-2*02 | D2- | J4*02 | SEQ ID | V4-69*01 | J3*02 | SEQ ID |
| | | (93%) | 15*01 | | NO: 12 | (94%) | | NO: 16 |
| PspA16 | IgG₁, k | V1-2*02 | D3- | J1*01 | SEQ ID | V3-20*01 | J3*01 | SEQ ID |
| | | (93%) | 10*01 | | NO: 36 | (94%) | | NO: 40 |

Analysis was performed using IMGT/V-Quest.

mAbs PhtD3 and PhtD6 utilize kappa light chains, while mAbs PhtD7 and PhtD8 use lambda light chains. All mAbs were of the IgG₁ isotype based on isotyping data determined by ELISA. All mAbs utilize unique heavy chain and light chain V genes, with the exception of mAbs PhtD7 and PhtD8. mAbs PhtD7 and PhtD8 share V_{H} and JH gene usage, although vary in the use of the DH gene, which leads to stark differences in CDR3 lengths, with mAbs PhtD7 and PhtD8 having 20 amino acid and 8 amino acid length HCDR3 lengths, respectively. mAbs PhtD7 and PhtD8 also share predicted V_{L} and J_{L} gene usage, although LCDR3 sequences share little sequence identity.

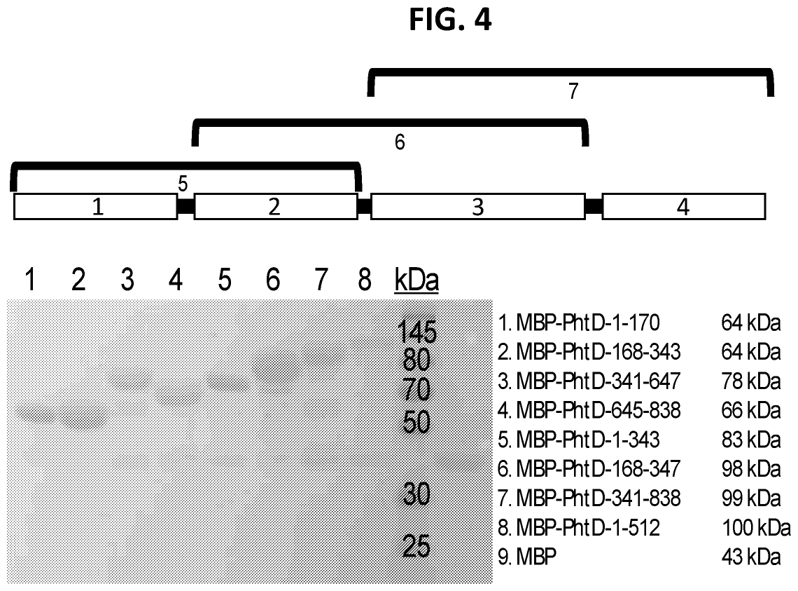

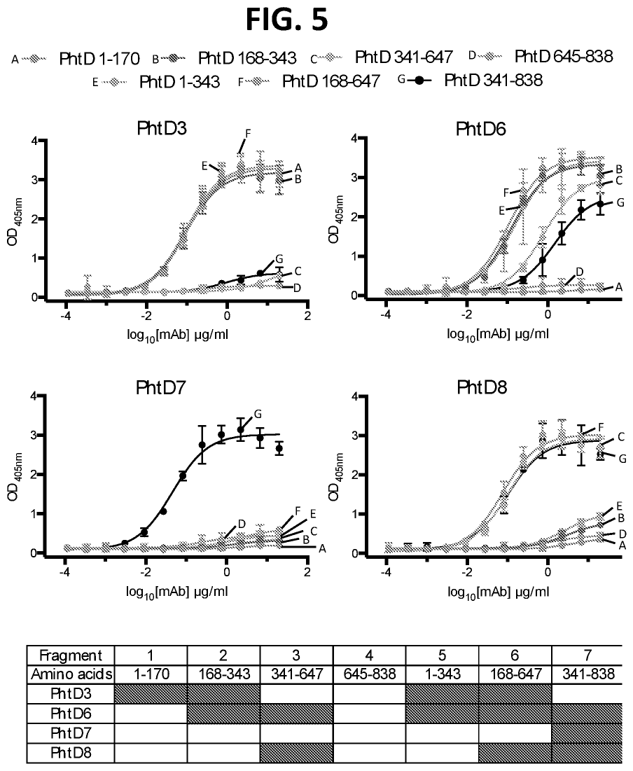

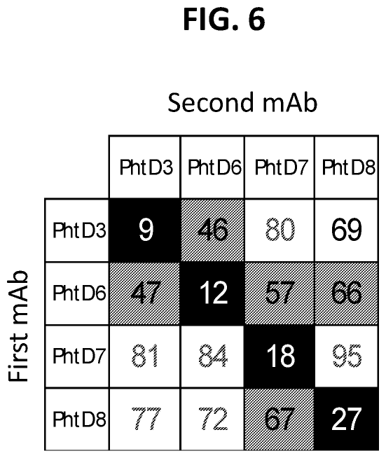

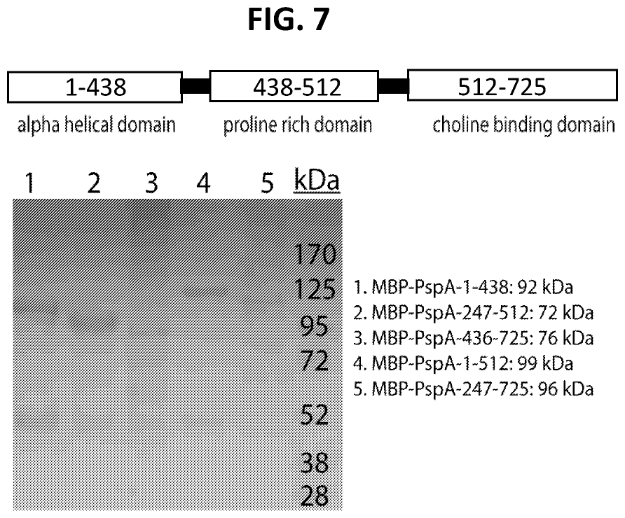

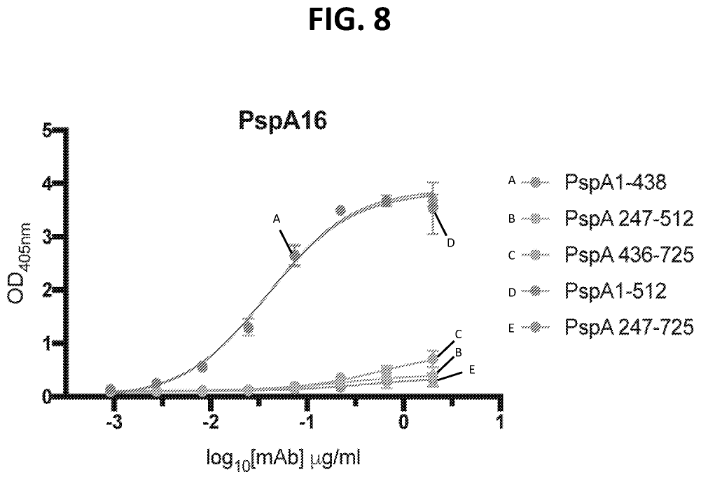

To identify the specific regions of PhtD targeted by the human mAbs, truncated fragments of PhtD were generated based on a secondary structure predictor. The fragments were fused to the maltose binding protein (MBP) to ensure solubility, and expressed in E. coli and purified using amylose resin. The majority of the fragments were >90% pure with the exception of free MBP protein for the MBP fusion proteins (FIG. 4). To identify the specific regions of PhtD targeted by the isolated mAbs, we measured ELISA binding of mAbs to fragments of PhtD. Since there are no previous mAbs that have been generated to these proteins with defined epitopes, the generated fragments provide rough estimates of mAb epitopes. Each of the four mAbs bind to a unique region on the PhtD protein (FIG. 5). mAbs PhtD3 and PhtD6 bind the N-terminal portion of the protein, while mAb PhtD8 binds the C-terminal portion. mAb PhtD7 appears to target a unique conformational epitope that is dependent on amino acids 341-838, but this mAb does not bind 341-647 or 645-838 fragments. The epitopes of the mAbs were assessed by competitive biolayer interferometry to compare the binding epitopes between mAbs (FIG. 6). Anti penta-His biosensors were loaded with His-tagged PhtD protein, and mAbs were competed for binding sequentially (FIG. 6). The mAbs bind distinct regions with limited competition similar to results from the fragment ELISA data. mAbs PhtD3 and PhtD6 show intermediate competition, and the epitopes for these mAbs also overlap in our fragment ELISA data. To map the binding region of mAb PspA16, PspA was fragmented into several truncations based on previously determined domains (FIG. 7) (Khan N, Jan A T. (2017) Front Microbiol 8:742). mAb PspA16 had high avidity to recombinant PspA, and bound to the N-terminal fragment 1-247 based on positive binding to amino acid fragments 1-438 and 1-512 and negative binding to 247-512, 436-725, and 247-725 fragments (FIG. 8).

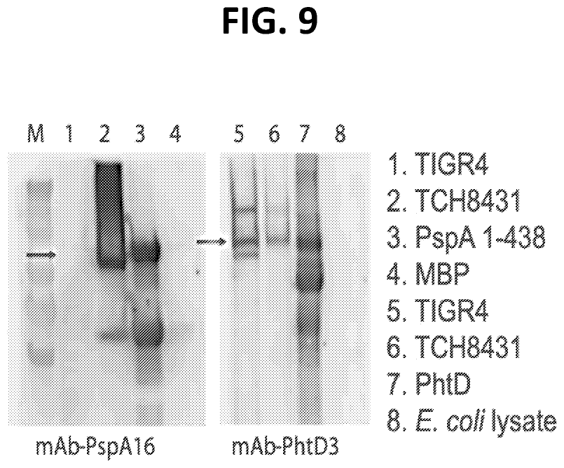

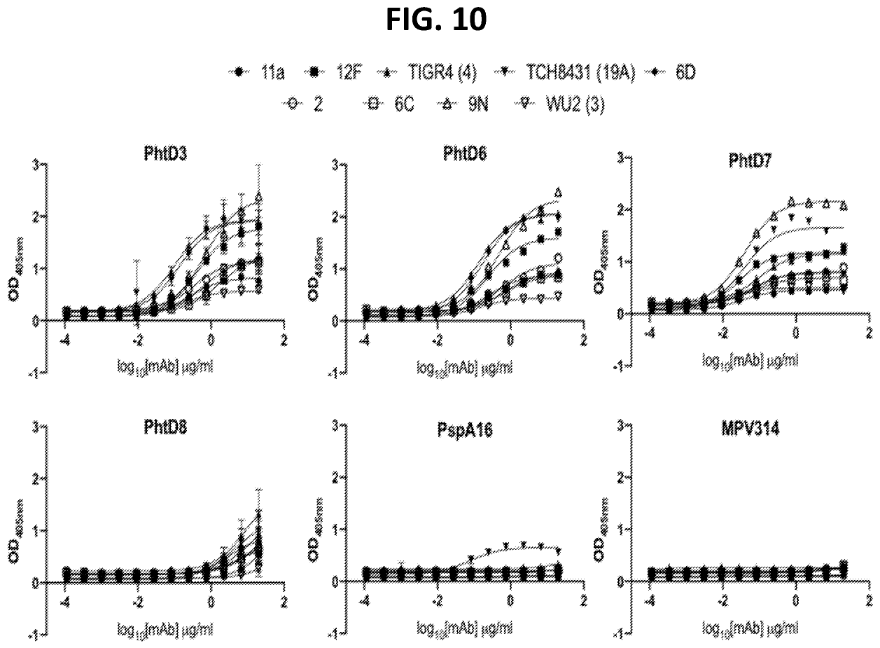

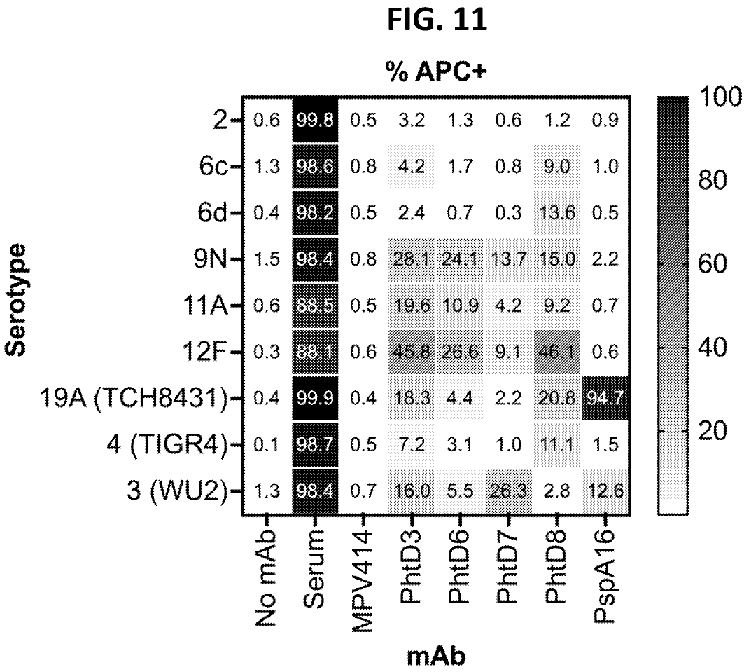

Pneumococcal surface proteins PhtD and PspA are conserved across serotypes, and are widely prevalent in the majority of serotypes. As such, human mAbs to these antigens could have the potential to treat pneumococcal infection from multiple serotypes. In order to determine the serotype breadth of the isolated mAbs, mAb binding was assessed to two diverse pneumococcal serotypes, strain TCH8431 (serotype 19A), from which the genes for recombinant PhtD and PspA proteins were cloned and expressed, and the commonly used laboratory strain TIGR4 (serotype 4). PspA shares 88% amino acid sequence identity between TCH8431 and TIGR4, although significant variability is present in the N-terminal domain, with 70% identity in amino acids 1-247. In contrast, PhtD shares 98% amino acid sequence identity between these two strains. Western blots were conducted by probing bacterial lysates from TIGR4 and TCH8431 with mAbs PhtD3 and PspA16. mAb PspA16 only labels PspA protein from strain TCH8431 (serotype 19A) (FIG. 9), while mAb PhtD3 is able to label PhtD protein from both pneumococcal strains. However, as the bacterial lysis likely results in protein denaturation, it is possible the epitope for mAb PspA16 is altered during denaturation. It was next determined if mAbs isolated against each of the recombinant proteins bind whole bacteria. ELISA assays were conducted by coating plates with fixed bacteria and measuring mAb binding by ELISA. mAbs PhtD 3, PhtD6, PhtD7, and PhtD8 were broadly reactive across multiple unrelated pneumococcal serotypes, and mAbs PhtD3, PhtD6, and PhtD7 had higher avidity to fixed bacteria as compared to PhtD8 (FIG. 10). In contrast, PspA16 bound only to strain TCH8431, similar to results from the western blot experiments. Since PspA16 binds to the most variable region of PspA, the reduced binding to divergent serotypes was expected. In a third experiment, binding of the mAbs to a panel of pneumococcal serotypes was assessed by flow cytometry. Serum was utilized from a donor vaccinated 21 days previously with Prevnar-13@ as a positive control. The PhtD mAbs bound to the majority of tested serotypes, with mAbs PhtD3 and PhtD8 showing the broadest binding (FIG. 11). In contrast, PspA16 bound only to TCH8431 and the serotype 3 strain WU2.

### Example 3

### mAB Treatment Protects Mice from Pneumococcal Infection

mAbs PhtD3, PhtD8, PspA16, and PhtD7 were further analyzed for protective efficacy in a mouse model of pneumococcal infection.

As mAbs PhtD3 and PhtD8 exhibited the highest overall breadth in the serotype binding analysis by flow cytometry, these mAbs were further analyzed for protective efficacy in the mouse model. In addition, these mAbs were chosen in order to identify if the epitope specificity of mAbs to PhtD affect protective efficacy, as they target nonoverlapping epitopes. Mouse mAbs to PhtD and polyclonal human antibodies from both healthy human subjects and PhtD-vaccinated humans have been shown to protect against colonization or disease in mouse models of pneumococcal infection. However, no human mAbs have been examined for protective efficacy.

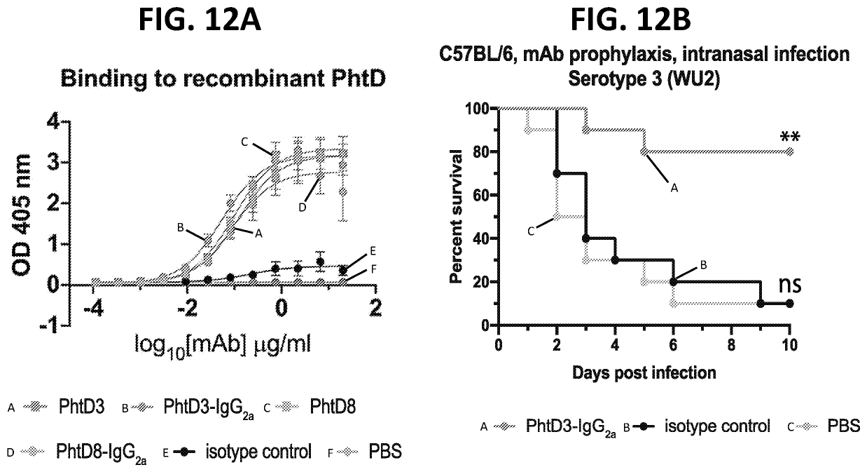

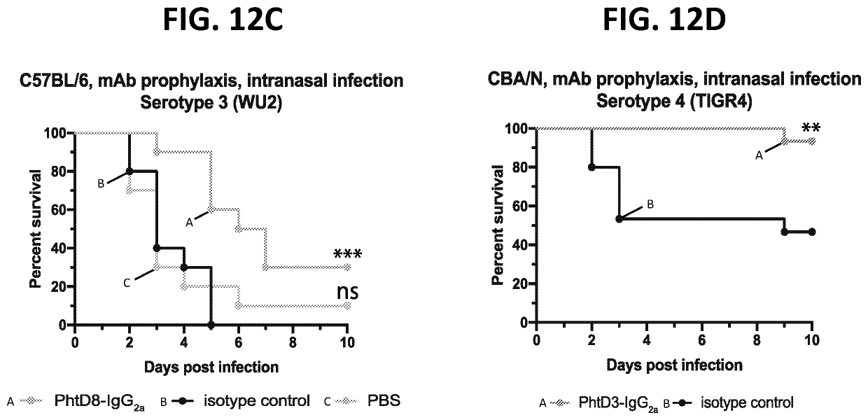

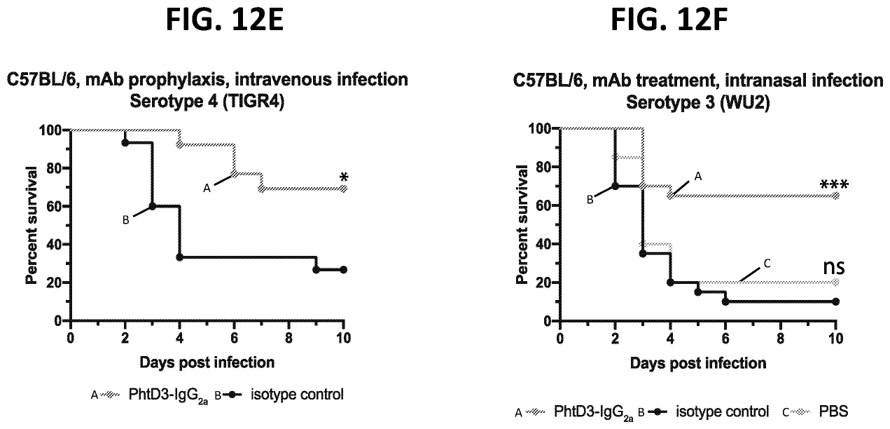

To determine if the PhtD-specific mAbs protect against infection, the efficacy of mAbs PhtD3 and PhtD8 were examined in a mouse model of pneumococcal pneumonia with a serotype 3 strain (WU2), as serotype 3 is a leading cause of invasive pneumococcal disease. Since the mAbs were isolated from human hybridomas, and thus have authentic human Fc regions, the Fc region was isotype-switched to the closest mouse homolog (human IgG₁ became mouse IgG₂ₐ). mAbs PhtD3 and PhtD8 chimeras with mouse IgG2a Fc regions (PhtD3-IgG2a and PhtD8-IgG2a) were recombinantly expressed in HEK293F cells for testing in the mouse model. As a control for the study, a mouse IgG₂ₐ isotype control antibody was purchased. The binding of the mAbs was first examined to ensure binding was still observed for the recombinant PhtD3-IgG₂ₐ and PhtD8-IgG₂ₐ mAbs, and that no binding was observed for the isotype control mAb. As expected, PhtD3-IgG₂ₐ and PhtD8-IgG₂ₐ had similar binding avidity to recombinant PhtD as hybridoma-derived PhtD, while the isotype control showed no binding (FIG. 12A).

The prophylactic efficacy of PhtD3-IgG₂ₐ and PhtD8-IgG₂ₐ was first tested in a pneumonia model with pneumococcal serotype 3. Both mAbs prolonged the survival of mice compared to the isotype control, although those mice treated with mAb PhtD3 demonstrated higher survival (80% versus 30%) (FIGS. 12B and 12C). As mAb PhtD3-IgG₂ₐ protected a larger percentage of mice, this mAb was selected for further analysis. mAb PhtD3-IgG₂ₐ was then tested for protective efficacy against pneumococcal serotype 4 (TIGR4) to identify if the broad binding correlates to broad protection. For this serotype, CBA/N mice were used for the intranasal infection model as TIGR4 was not sufficiently lethal by intranasal infection in C57BL/6 mice. CBA/N mice have previously been shown to be susceptible to serotype 4 (Sandgren et al. (2005) J Infect Dis 192:791-800). PhtD3-IgG₂ₐ prolonged survival of mice, providing 93% protection compared to 47% to the isotype control (FIG. 12D). As it was not possible to test PhtD3-IgG₂ₐ in an intranasal infection model with TIGR4 in C57BL/6 mice, an experiment was conducted in C57BL/6 mice in which mice were intravenously infected with TIGR4 to model septic pneumococcal infection. In this study, PhtD3-IgG2a prolonged survival of mice with 69% efficacy compared to 27% survival with the isotype control (FIG. 12E). The most clinically relevant scenario for mAb treatment of pneumococcal infection would be administration after pneumococcal infection. To model such a scenario, mice were infected with pneumococcal serotype 3, and administered mAb PhtD3-IgG₂ₐ 24 hrs after infection. In this model, 65% of PhtD3-IgG₂ₐ treated mice survived the infection compared to 10% for the isotype control group (FIG. 12F).

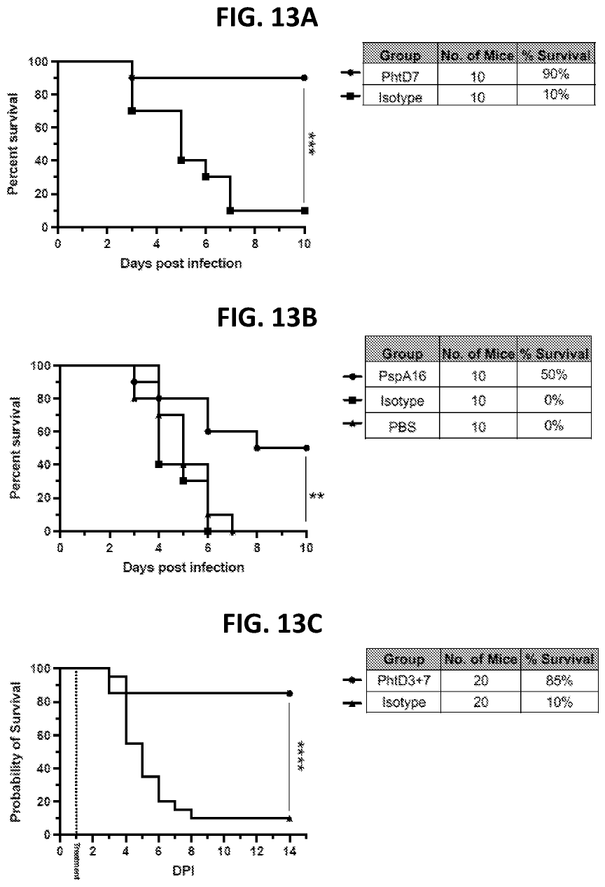

Since the above data indicates that mAbs targeting the pneumococcal histidine triad protein D (PhtD) are protective, mAb PhtD7, which targets a different region of PhtD, was evaluated to determine if it is also protective. Two groups of mice (n=10/group) were infected intranasally with 5×10⁶ CFU of serotype 3 (WU2) Streptococcus pneumoniae. Two hours before infection, mice were administered PhtD7 (fully human; 15 mg/kg) or isotype control IgG₂ₐ (15 mg/kg) via intraperitoneal injection. Survival of mice was monitored over time and expressed as a percent (FIG. 13A). PhtD7 was found to have a significant protective effect, showing an 80% increase in survival as compared to the control.

In addition, mAb PspA16, which targets Pneumococcal surface protein A (PspA), was also determined to have a significant protective effect in a murine serotype 3 intranasal infection model. Three groups of C57BL/6 mice (n=10/group) were infected intranasally with 5×10⁶ CFU of serotype 3 (WU2) Streptococcus pneumoniae. Two hours before infection, mice were administered PspA16 (fully human; 15 mg/kg), isotype control IgG₂ₐ (15 mg/kg), or PBS via intraperitoneal injection. Survival of mice was monitored over time and expressed as a percent (FIG. 13B). PspA16 treatment was found to have a significant protective effect with a 50% increase in survival compared to isotype and PBS controls.

### Example 4

### Co-Treatment with mABs PhtD3 and PhtD7 Post Infection Increases Survival

Since the above data demonstrated a protective effect for both mAbs PhtD3 and PhtD7, it was determined whether a combination of both mAbs would have a protective effect when administered post-infection as opposed to pre-infection. Two groups of mice (n=20/group) were infected intranasally with 5×10⁶ CFU of serotype 3 (WU2) Streptococcus pneumoniae. 24 hours post infection mice were administered a combination of PhtD3-IgG₂ₐ (labeled as PhtD3) and PhtD7 (7.5 mg/kg each) or isotype control IgG₂ₐ (15 mg/kg), via intraperitoneal injection. Survival of mice was monitored over time and expressed as a percent (FIG. 13C).

The combination of both mAbs delivered IP 24 hrs after intranasal infection had a significant effect, with a 75% increase in survival compared to the isotype control (FIG. 13C).

### Example 5

### PhtD-Specific Human mAbs have Opsonophagocytic Activity

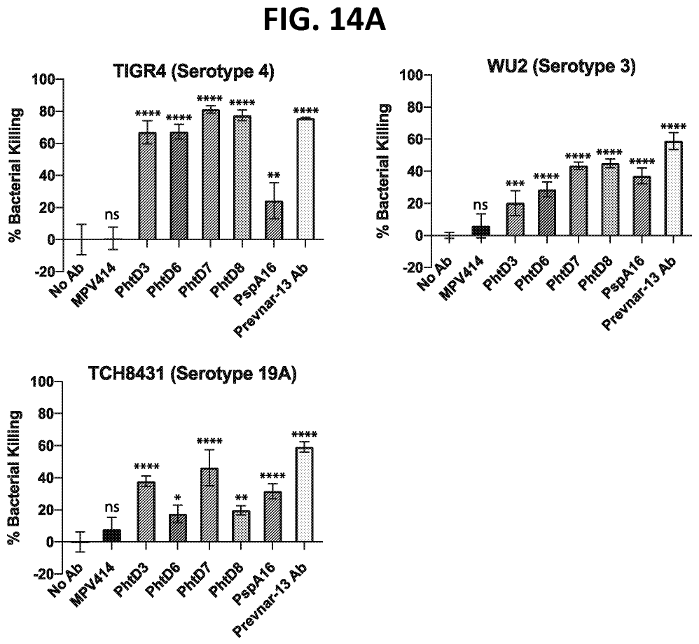

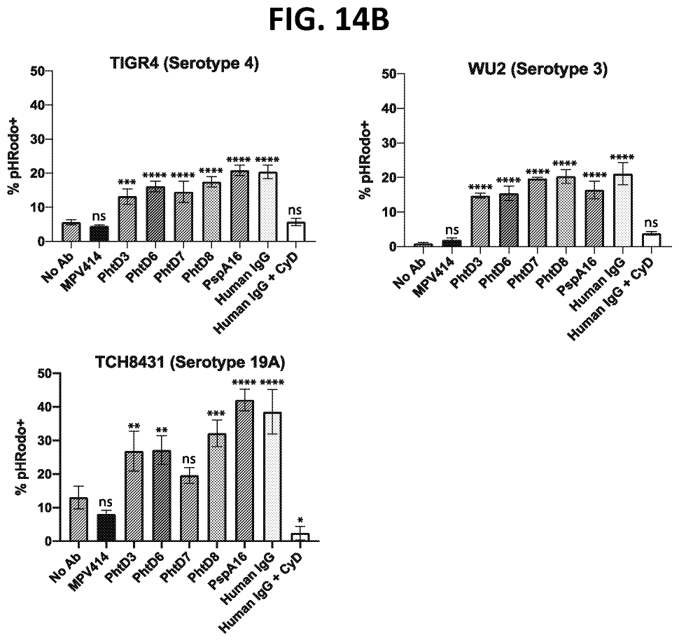

The correlate of protection for current pneumococcal vaccines is based on the elicitation of anti-capsule antibodies that opsonize bacteria, leading to their phagocytosis by host immune cells and subsequent bacterial killing (Romero-Steiner et al. (2006) Clin Vaccine Immunol 13:165-169; Paschall et al. (2019) JoVE e59400). Mouse mAbs isolated by vaccination with PhtD were previously shown to induce bacterial opsonophagocytosis, which was dependent on complement and macrophages (Visan et al. (2018) Hum Vaccin Immunother 14:489-494). To determine a potential mechanism of protection by PhtD3, and additional PhtD mAbs, established opsonophagocytosis killing assays (OPKAs) using the HL-60 cell line were utilized. The mAbs were tested against serotypes 4 (strain TIGR4), 3 (strain WU2), and serotype 19A (strain TCH8431), from which the PhtD and PspA constructs were cloned. These mAbs were also compared to purified IgG obtained from a human subject previously vaccinated with Prevnar-13@21 days before blood collection, as the OPKA assay is the standard to measure vaccine uptake (Pilishvili (2015) Vaccine 33:D60-D65). All PhtD mAbs induced decreased colony forming units against all three serotypes compared to no antibody and an irrelevant mAb to human metapneumovirus (FIG. 14A). PspA16 also decreased colony forming units against all three serotypes, although the efficacy against serotype 4 was lower as expected based on the serotype binding data. To confirm these findings, a flow-based assay was adopted that was previously shown to work for group B Streptococcus (Fabbrini et al. (2012) J Immunol Methods 378:11-19). HL-60 cells were incubated with opsonized bacteria that were labeled with pHRodo™, which leads to fluorescent HL-60 cells upon phagocytosis of labeled bacteria. Similar to our results from the OPA assay, all PhtD mAbs induced an increase in pHRodo™+HL-60 cells compared to a no antibody and isotype control antibody (FIG. 14B). Purified IgG from an unvaccinated donor showed the highest number of pHRodo™+ cells, as human IgG contains antibodies to multiple pneumococcal surface proteins. Interestingly, PspA16 also induced increased uptake to all three serotypes in this assay, although the highest activity was observed for serotype 19A, the serotype from which the PspA gene was cloned.

### Example 6

### mAb PhtD3 Treatment Reduces Lung and Blood Bacterial Titers

The following describes the effect of mAb PhtD3-IgG₂ₐ treatment on lung and blood bacterial titers 3 days post-infection.

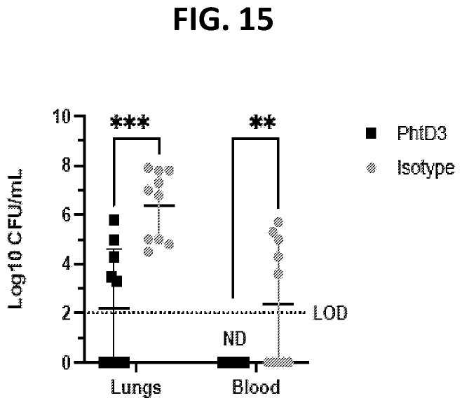

Two groups of mice (n=10/group) were infected intranasally with 5×10⁶ CFU of serotype 3 (WU2) Streptococcus pneumoniae. Two hours before infection, mice were administered PhtD3-IgG2a (labeled PhtD3; 15 mg/kg) or isotype control IgG2a (15 mg/kg) via intraperitoneal injection. Three days post-infection, mice were euthanized, and lungs and blood were collected. Lungs were homogenized, and lung homogenates and blood were serially diluted and titered on sheep blood agar plates. Data is represented as log₁₀ of the CFU/mL of lung homogenates or blood (FIG. 15). Statistical comparison for lung and blood titers using an unpaired t-test showed a p-value of 0.0002 and 0.009, respectively, when compared to isotype control groups.

Lung titers showed about a 4-log reduction compared to isotype control, with some treated animals having titers below the limit of detection. Blood titers showed similar results, with about a 2.4-log reduction as compared to isotype control, with no PhtD3 treated animals showing detectable levels of bacteria in the blood (see, FIG. 15).

### Example 7

### mAb PhtD3 is Protective in an Influenza Co-Infection Model

The following describes the protective effect of mAb PhtD3-IgG₂ₐ treatment in an influenza co-infection model.

Three groups of mice (n=20/group) were infected intranasally with 100 FFU of Influenza A virus CA04/09 (IAV). Seven days post-influenza infection, mice were intranasally infected with 1×10⁴ CFU of serotype 3 (WU2) Streptococcus pneumoniae. Two hours before S. pneumoniae infection, mice were administered PhtD3-IgG₂ₐ (labeled PhtD3; 15 mg/kg), Isotype control IgG2a (15 mg/kg), or PBS via intraperitoneal injection. Two groups of mice (n=5/group) were singly infected with IAV or S. pneumoniae to ensure single infections alone were not lethal.

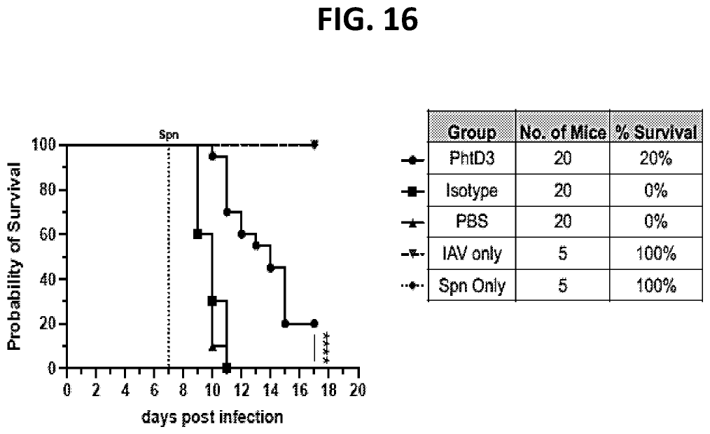

Survival of mice was monitored over time and expressed as a percent (FIG. 16). Statistical comparison of survival curves of PhtD3 vs isotype control groups using log-ranked (Mantel-Cox) test showed a p-value of <0.0001. Mice treated with PhtD3 had a 20% increase in survival compared to controls, and the time to mortality was significantly extended compared to controls.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isolated monoclonal antibody or antigen binding fragment thereof, comprising:
a) a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as SEQ ID NOs: 1 and 5, respectively;
b) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 25 and 29, respectively;
c) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 9 and 13, respectively;
d) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 17 and 21, respectively; or
e) a V_{H} and a V_{L} comprising a HCDR1, a HCDR2, and a HCDR3, and a LCDR1, a LCDR2, and a LCDR3 of the V_{H} and V_{L} set forth as SEQ ID NOs: 33 and 37, respectively, wherein the monoclonal antibody specifically binds Streptococcus pneumoniae.

2. The isolated antibody or antigen binding fragment of claim 1, wherein
a) the HCDR1, the HCDR2, the HCDR3, the LCDR1, the LCDR2, and the LCDR3 comprise the amino acids sequences set forth as SEQ ID NOs: 2, 3, 4, 6, 7, and 8, respectively;
b) the HCDR1, the HCDR2, the HCDR3, the LCDR1, the LCDR2, and the LCDR3 comprise the amino acids sequences set forth as SEQ ID NOs: 26, 27, 28, 30, 31, and 32 respectively;
c) the HCDR1, the HCDR2, the HCDR3, the LCDR1, the LCDR2, and the LCDR3 comprise the amino acids sequences set forth as SEQ ID NOs: 10, 11, 12, 14, 15, and 16, respectively;
d) the HCDR1, the HCDR2, the HCDR3, the LCDR1, the LCDR2, and the LCDR3 comprise the amino acids sequences set forth as SEQ ID NOs: 18, 19, 20, 22, 23, and 24 respectively; or
e) the HCDR1, the HCDR2, the HCDR3, the LCDR1, the LCDR2, and the LCDR3 comprise the amino acids sequences set forth as SEQ ID NOs: 34, 35, 36, 38, 39, and 40 respectively.

3. The isolated antibody or antigen binding fragment of claim 1, wherein
a) the V_{H} and the V_{L} comprise the amino acid sequences at least 90% identical to the amino acid sequences set forth as SEQ ID NOs: 1 and 5, respectively;
b) the V_{H} and the V_{L} comprise the amino acid sequences at least 90% identical to the amino acid sequences set forth as SEQ ID NOs: 25 and 29, respectively; or
c) the V_{H} and the V_{L} comprise the amino acid sequences at least 90% identical to the amino acid sequences set forth as SEQ ID NOs: 9 and 13, respectively;
d) the V_{H} and the V_{L} comprise the amino acid sequences at least 90% identical to the amino acid sequences set forth as SEQ ID NOs: 17 and 21, respectively; or
e) the V_{H} and the V_{L} comprise the amino acid sequences at least 90% identical to the amino acid sequences set forth as SEQ ID NOs: 33 and 37, respectively.

4. The isolated antibody or antigen binding fragment of claim 1, comprising a human framework region.

5. The isolated antibody or antigen binding fragment of claim 1, wherein:
a) the V_{H} and the V_{L} comprise the amino acid sequences set forth as SEQ ID NOs: 1 and 5, respectively;
b) the V_{H} and the V_{L} comprise the amino acid sequences set forth as SEQ ID NOs: 25 and 29, respectively;
c) the V_{H} and the V_{L} comprise the amino acid sequences set forth as SEQ ID NOs: 9 and 13, respectively;
d) the V_{H} and the V_{L} comprise the amino acid sequences set forth as SEQ ID NOs: 17 and 21, respectively; or
e) the V_{H} and the V_{L} comprise the amino acid sequences set forth as SEQ ID NOs: 33 and 37, respectively.

6-7. (canceled)

8. The isolated antibody of claim 1, comprising a recombinant constant domain comprising a modification that increases the half-life of the antibody.

9. The isolated antibody of claim 8, wherein the modification increases binding to the neonatal Fc receptor.

10. The isolated antibody or antigen binding fragment of claim 1,
wherein the antibody or antigen binding fragment comprises the HCDR1, the HCDR2, the HCDR3, the LCDR1, the LCDR2, and the LCDR3 of the V_{H} and V_{L} set forth as
a) SEQ ID NOs: 1 and 5, respectively,
b) SEQ ID NOs: 25 and 29, respectively,
c) SEQ ID NOs: 9 and 13, respectively, or
d) SEQ ID NOs: 17 and 21, respectively; and
wherein the antibody or antigen binding fragment specifically binds pneumococcal histidine triad protein (PhtD).

11. The isolated antibody or antigen binding fragment of claim 1,
wherein the antibody or antigen binding fragment comprises the HCDR1, the HCDR2, the HCDR3, the LCDR1, the LCDR2, and the LCDR3 of the V_{H} and V_{L} set forth as SEQ ID NOs: 33 and 37, respectively; and wherein the antibody or antigen binding fragment specifically binds pneumococcal surface protein A (PspA).

12. The isolated antigen binding fragment of claim 1.

13. The isolated antigen binding fragment of claim 12, wherein the antigen binding fragment is a Fv, Fab, F(ab′)₂, scFV or a scFV₂ fragment.

14. (canceled)

15. A multi-specific antibody comprising the antibody or antigen binding fragment of claim 1.

16. An isolated nucleic acid molecule encoding the antibody or antigen binding fragment of claim 1.

17. (canceled)

18. The nucleic acid molecule of claim 16, operably linked to a promoter.

19. A vector comprising the nucleic acid molecule of claim 16.

20. A host cell comprising the vector of claim 19.

21. A pharmaceutical composition comprising an effective amount of the antibody or antigen binding fragment of claim 1; and
a pharmaceutically acceptable carrier.

22. A method of producing an antibody or antigen binding fragment that specifically binds to PhtD or PspA, comprising:
expressing one or more nucleic acid molecules encoding the antibody; or antigen binding fragment of claim 1 in a host cell; and
purifying the antibody or antigen binding fragment.

23. A method of detecting the presence of a Streptococcus pneumoniae in a biological sample from a subject, comprising:
contacting the biological sample with an effective amount of the antibody or antigen binding fragment of claim 1 under conditions sufficient to form an immune complex; and
detecting the presence of the immune complex in the biological sample, wherein the presence of the immune complex in the biological sample indicates the presence of the Streptococcus pneumoniae in the sample.

24. (canceled)

25. A method of inhibiting a Streptococcus pneumoniae infection in a subject, comprising administering an effective amount of the antibody or antigen binding fragment of claim 1 to the subject, wherein the subject has or is at risk of a Streptococcus pneumoniae infection.

26-33. (canceled)


SEQUENCE LISTING (64 sequences)

SEQ ID NO: 1 - protein, 125 aa, Artificial Sequence
  PhtD3 VH
QVQLVQSGPDVKKPGSSVKVSCKASGAAFESFAFAWVRQAPGQGFEWMGRIIPILETRDYAEKFQGRMTMTTDESTATAYMELNSLRFEDTAVYFCARDGHIMRTTLSDAALDVWGQGTTVIVSS

SEQ ID NO: 2 - protein, 8 aa, Artificial Sequence
  PhtD3 HCDR1
GAAFESFA

SEQ ID NO: 3 - protein, 8 aa, Artificial Sequence
  PhtD3 HCDR2
IIPILETR

SEQ ID NO: 4 - protein, 18 aa, Artificial Sequence
  PhtD3 HCDR3
ARDGHIMRTTLSDAALDV

SEQ ID NO: 5 - protein, 108 aa, Artificial Sequence
  PhtD3 VL
DIVMTQSPVTLSLSPGERATLSCRASQSLTDNYLAWYQQKPGQAPRLLIYAASTRATGIPDRISGSGSGTDFTLTISRVEPEDFAMFYCQQYQNSPFTFGGGTTVEIK

SEQ ID NO: 6 - protein, 7 aa, Artificial Sequence
  PhtD3 LCDR1
QSLTDNY

SEQ ID NO: 7 - protein, 3 aa, Artificial Sequence
  PhtD3 LCDR2
AAS

SEQ ID NO: 8 - protein, 9 aa, Artificial Sequence
  PhtD3 LCDR3
QQYQNSPFT

SEQ ID NO: 9 - protein, 115 aa, Artificial Sequence
  PhtD8 VH
QVQLVQSGAEVKKPGASVKVSCKASGYTFTDYFIHWVRQAPGHGLEWMGWINPNRGVTNYTQKFQGRVTMTKDTSVTSVYMELSRLTSDDTALYYCARGGTLDHWGQGTLVTVSS

SEQ ID NO: 10 - protein, 8 aa, Artificial Sequence
  PhtD8 HCDR1
GYTFTDYF

SEQ ID NO: 11 - protein, 8 aa, Artificial Sequence
  PhtD8 HCDR2
INPNRGVT

SEQ ID NO: 12 - protein, 8 aa, Artificial Sequence
  PhtD8 HCDR3
ARGGTLDH

SEQ ID NO: 13 - protein, 112 aa, Artificial Sequence
  PhtD8 VL
QLVLTQSPSASASLGASVTLTCTLSSGHSTYDIAWHQQQPGKGPRHLMRLNGDGSHTNGDGIPDRFSGSSSGAERYLTISSLQSEDEADYYCHTWVTNIHLVFGGGTKLTVL

SEQ ID NO: 14 - protein, 7 aa, Artificial Sequence
  PhtD8 LCDR1
SGHSTYD

SEQ ID NO: 15 - protein, 7 aa, Artificial Sequence
  PhtD8 LCDR2
LNGDGSH

SEQ ID NO: 16 - protein, 10 aa, Artificial Sequence
  PhtD8 LCDR3
HTWVTNIHLV

SEQ ID NO: 17 - protein, 120 aa, Artificial Sequence
  PhtD6 VH
QVQLVQSGTEVKKPGASVKVACKASGYTFTSYDINWVRQAPGQGLEWMGWMNANSGNTGYAQKFQGRVTMTRNTSITTAYMDLIDLTSEDTAIYYCARGPYWVENWFDTWGQGTLVSVSS

SEQ ID NO: 18 - protein, 8 aa, Artificial Sequence
  PhtD6 HCDR1
GYTFTSYD

SEQ ID NO: 19 - protein, 8 aa, Artificial Sequence
  PhtD6 HCDR2
MNANSGNT

SEQ ID NO: 20 - protein, 13 aa, Artificial Sequence
  PhtD6 HCDR3
ARGPYWVENWFDT

SEQ ID NO: 21 - protein, 107 aa, Artificial Sequence
  PhtD6 VL
DIQMTQSPSSLSASVGDRVTITCRASRSIRSFLNWYQQKPGKPPNLLIYKASTLHSGVPSRFSGSGSGTDFTLTINNLQPEDFATYYCQQSYSNQKTFGQGTKVDIK

SEQ ID NO: 22 - protein, 6 aa, Artificial Sequence
  PhtD6 LCDR1
RSIRSF

SEQ ID NO: 23 - protein, 3 aa, Artificial Sequence
  PhtD6 LCDR2
KAS

SEQ ID NO: 24 - protein, 9 aa, Artificial Sequence
  PhtD6 LCDR3
QQSYSNQKT

SEQ ID NO: 25 - protein, 127 aa, Artificial Sequence
  PhtD7 VH
EVQLVQSGAEVKKPGASVKVSCKASGDIFSDSYIHWVRQAPGQGPEWMGWVSPNTGATHYAQKLQGRVTMTSDTSISTAYLELTRLASDDTAVYYCARVLRGSYDFRGNYPHDFDYWGQGTLVTVSS

SEQ ID NO: 26 - protein, 8 aa, Artificial Sequence
  PhtD7 HCDR1
GDIFSDSY

SEQ ID NO: 27 - protein, 8 aa, Artificial Sequence
  PhtD7 HCDR2
VSPNTGAT

SEQ ID NO: 28 - protein, 20 aa, Artificial Sequence
  PhtD7 HCDR3
ARVLRGSYDFRGNYPHDFDY

SEQ ID NO: 29 - protein, 112 aa, Artificial Sequence
  PhtD7 VL
QLVLTQPPSASASLGASVTLTCTLSRGHNNYPIAWLQKQTDKGPRYVMRLNSDGSHHKGDGIPDRFSGSSSGAERYLSISSLQPEDEAEYYCQTWDTGLQGVFGGGTKLFVL

SEQ ID NO: 30 - protein, 7 aa, Artificial Sequence
  PhtD7 LCDR1
RGHNNYP

SEQ ID NO: 31 - protein, 7 aa, Artificial Sequence
  PhtD7 LCDR2
LNSDGSH

SEQ ID NO: 32 - protein, 10 aa, Artificial Sequence
  PhtD7 LCDR3
QTWDTGLQGV

SEQ ID NO: 33 - protein, 120 aa, Artificial Sequence
  PspA16 VH
QVQLVQSGPDVKKPGASVKVSCKTSGYTFTGYYMHWVRQAPGQGLEWMGWVNPNTGGTSYAQKFQGRVTVTRDTSISTVYMELSALGSDDTAIYFCARAWAPGAEYLHHWGQGTLVTVSS

SEQ ID NO: 34 - protein, 8 aa, Artificial Sequence
  PspA16 HCDR1
GYTFTGYY

SEQ ID NO: 35 - protein, 8 aa, Artificial Sequence
  PspA16 HCDR2
VNPNTGGT

SEQ ID NO: 36 - protein, 13 aa, Artificial Sequence
  PspA16 HCDR3
ARAWAPGAEYLHH

SEQ ID NO: 37 - protein, 108 aa, Artificial Sequence
  PspA16 VL
EIVMTQSPGTLSLSPGETATLSCRASQSVGSSYLAWYQQKPGQAPRLLIFGASNRATGIPVRFSASGSGTDFTLTISRLEPEDFAVYYCQQHDHSPFTFGPGTKVDLK

SEQ ID NO: 38 - protein, 7 aa, Artificial Sequence
  PspA16 LCDR1
QSVGSSY

SEQ ID NO: 39 - protein, 3 aa, Artificial Sequence
  PspA16 LCDR2
GAS

SEQ ID NO: 40 - protein, 9 aa, Artificial Sequence
  PspA16 LCDR3
QQHDHSPFT

SEQ ID NO: 41 - DNA, 376 bp, Artificial Sequence
  PhtD3 VH
caggtgcagctagtgcagtctgggcctgacgtgaagaagcctgggtcctcggtgaaggtctcctgcaaggcctctggagccgccttcgagagttttgccttcgcctgggtgcgacaggcccctggacaagggtttgagtggatgggaaggatcattccaatcttggaaacaagggactacgcagagaagttccagggcagaatgacgatgaccacagacgagtcgacggcgacagcctacatggaactgaacagcctaagatttgaagacacggccgtttatttctgtgcgcgagatgggcacattatgaggacaactctctcggatgctgcacttgatgtctggggccaagggacaacggtcattgtctcctcag

SEQ ID NO: 42 - DNA, 325 bp, Artificial Sequence
  PhtD3 VL
gacatcgtgatgacccagtctccagtcaccctgtctttgtctccaggggagagagccaccctctcctgcagggccagtcagagtcttactgacaactacttagcctggtaccagcagaaacctggccaggctcccaggctcctcatctacgccgcatccaccagggccactggcatcccagacaggatcagtggcagtgggtctgggacagacttcactctcaccatcagcagagtggagcctgaagattttgcaatgttttactgtcaacagtatcagaactcaccgttcaccttcggcggggggaccacggtggagatcaaac

SEQ ID NO: 43 - DNA, 346 bp, Artificial Sequence
  PhtD8 VH
caggtgcagctggtgcagtctggggctgaggtgaagaagcctggggcctcagtgaaggtctcctgtaaggcttctggatacaccttcaccgactactttatacactgggtgcgacaggcccctggacacggtcttgaatggatggggtggatcaaccctaaccgcggtgtcacaaactatacacagaagtttcagggcagggtcaccatgaccaaggacacgtccgtcacctcagtctacatggagctgagcaggctgacatctgacgacacggccctatattattgtgcgagaggtggtacgcttgaccactggggccagggcaccctggtcaccgtctcctctg

SEQ ID NO: 44 - DNA, 337 bp, Artificial Sequence
  PhtD8 VL
cagcttgtgctgactcaatcgccctctgcctctgcctccctgggagcctcggtcaccctcacctgcactctgagcagtgggcacagcacctacgacatcgcatggcatcagcagcagccaggaaagggccctcgacacttgatgagacttaacggtgatggcagtcacaccaacggggacgggatccctgatcgcttctcaggctccagctctggggctgagcgctacctcaccatctccagcctccagtctgaagatgaggctgactattactgtcacacctgggtcactaacattcatttggtgttcggcggagggaccaaactgaccgtcctag

SEQ ID NO: 45 - DNA, 361 bp, Artificial Sequence
  PhtD6 VH
caggtgcagttggtgcagtctgggactgaggtgaagaagcctggggcctcagtgaaggtcgcctgcaaggcttctggatacaccttcactagttatgatatcaactgggtgcgacaggcccctggacaagggcttgagtggatgggatggatgaacgcgaacagtggcaacacaggctatgcacaaaagttccagggcagagtcaccatgaccaggaacacctccattaccacagcctacatggacctgattgatctgacatctgaggacacggccatatattactgtgcgagagggccgtactgggtggagaattggttcgacacctggggccagggaaccctggtcagcgtctcctcag

SEQ ID NO: 46 - DNA, 322 bp, Artificial Sequence
  PhtD6 VL
gacatccagatgacccagtctccatcctccctgtctgcgtctgtcggagacagagtcaccatcacttgccgggcaagtcggagcattcgcagctttttaaattggtatcaacaaaaaccagggaaaccccctaacctcctgatctataaagcatccactttgcacagtggggtcccgtctaggttcagtggcagtggatctgggacagatttcactctcacaatcaacaatctacaacccgaagattttgcaacttactactgtcaacagagttacagtaatcagaagaccttcggccaagggaccaaggtggacatcaaac

SEQ ID NO: 47 - DNA, 382 bp, Artificial Sequence
  PhtD7 VH
gaggtgcagctggtgcagtctggggctgaagtgaagaagcctggggcctcagtgaaggtctcctgcaaggcttctggagacatcttcagcgactcctatattcactgggtgcgacaggcccctggacaagggcctgagtggatgggatgggtcagccctaacactggtgccacacattatgcacagaagttgcagggcagagtcaccatgaccagcgacacgtccatcagtacagcctatttggagctgaccaggctggcatctgacgacacggccgtttattactgtgcgagagtcttaaggggaagttatgatttccggggtaattatccacatgattttgactactggggccagggaaccctggtcaccgtctcctcag

SEQ ID NO: 48 - DNA, 337 bp, Artificial Sequence
  PhtD7 VL
cagcttgtgctgactcaaccgccctctgcctctgcctccctgggagcctcggtcaccctcacctgcactctgagcagaggacacaacaactaccccatcgcttggctccaaaagcagacagataagggccctcgttatgtgatgagacttaatagtgatggcagccaccacaagggggacggaatccctgatcgcttctcaggctccagttctggggctgagcgctacctcagcatttccagtctccagcctgaagatgaggctgaatactactgtcagacgtgggacactggccttcagggggtgttcggcggagggaccaaactgttcgtcctag

SEQ ID NO: 49 - DNA, 361 bp, Artificial Sequence
  PspA16 VH
caggtgcagctggtgcagtctgggcctgacgtgaagaagcctggggcctcagtgaaggtctcctgtaagacttctggatacaccttcactggctactatatgcactgggtgcgacaggcccctggacaagggcttgagtggatgggatgggtcaaccctaacaccggtggcacaagttatgcacagaagtttcagggcagggtcaccgtgaccagggacacgtccatcagcacagtctacatggaactgagcgctctaggatctgacgacacggccatatatttctgtgcgagggcgtgggctccgggcgctgagtacctccaccactggggccagggcaccctggtcaccgtctcctcag

SEQ ID NO: 50 - DNA, 325 bp, Artificial Sequence
  PspA16 VL
gagattgtgatgacccagtctccaggcaccctgtctttgtctccaggggaaacagccaccctctcctgcagggccagtcagagtgttggcagcagctacttagcctggtatcagcagaaacctggccaggctcccaggctcctcatctttggtgcgtccaacagggccactggcatcccagtcaggttcagtgccagtgggtctgggacagacttcactctcaccatcagcagactggagcctgaagatttcgcagtgtattactgtcagcagcatgatcactcaccattcactttcggccctgggaccaaagtggatctcaaac

SEQ ID NO: 51 - DNA, 35 bp, Artificial Sequence
  PspA1-F primer
cgccatatgatggctaataagaaaaaaatgatttt

SEQ ID NO: 52 - DNA, 27 bp, Artificial Sequence
  PspA247-F primer
cgccatatggagctaaacgctaaacaa

SEQ ID NO: 53 - DNA, 30 bp, Artificial Sequence
  PspA436-F primer
cgccatatggatgaagaagaaactccagcg

SEQ ID NO: 54 - DNA, 50 bp, Artificial Sequence
  PspA438-R primer
tagcggccgttaatggtgatggtgatggtgttcttcatctccatcagggc

SEQ ID NO: 55 - DNA, 51 bp, Artificial Sequence
  PspA512-R primer
tagcggccgttaatggtgatggtgatggtgttttggagtggctggtttttc

SEQ ID NO: 56 - DNA, 50 bp, Artificial Sequence
  PspA725-R primer
tagcggccgttaatggtgatggtgatggtgaacccattcaccattggcat

SEQ ID NO: 57 - DNA, 41 bp, Artificial Sequence
  PhtD1-F primer
catgccatggccatgaaaatcaataaaaaatatctagcagg

SEQ ID NO: 58 - DNA, 31 bp, Artificial Sequence
  PhtD168-F primer
catgccatggccgcagataatgctgttgctg

SEQ ID NO: 59 - DNA, 32 bp, Artificial Sequence
  PhtD341-F primer
catgccatggcctatcgttcaaaccattgggt

SEQ ID NO: 60 - DNA, 37 bp, Artificial Sequence
  PhtD645-F primer
catgccatggccgaccattaccataacatcaaatttg

SEQ ID NO: 61 - DNA, 57 bp, Artificial Sequence
  PhtD170-R
cccaagcttttaatggtgatggtgatggtgattatctgctcttgagttatgattatg

SEQ ID NO: 62 - DNA, 51 bp, Artificial Sequence
  PhtD343-R primer
cccaagcttttaatggtgatggtgatggtgtgaacgataacgaaggggaat

SEQ ID NO: 63 - DNA, 58 bp, Artificial Sequence
  PhtD647-R primer
cccaagcttttaatggtgatggtgatggtggtaatggtcataatgaggtatgattaaa

SEQ ID NO: 64 - DNA, 49 bp, Artificial Sequence
  PhtD838-R primer
cccaagcttttaatggtgatggtgatggtgctgtataggagccggttga